(19) United States
(12) Patent Application Publication
(10) Pub. No.: US 2021/0254972 A1
(43) Pub. Date: Aug. 19, 2021

(54) SYSTEM AND METHOD FOR ULTRASONIC DETECTION OF BIOFILM

(71) Applicant: UWM Research Foundation, Inc., Milwaukee, WI (US)

(72) Inventors: Marcia Renata Quadros da Silva, Oak Creek, WI (US); Sachin Davis, Franklin, WI (US); Adibi Yasaman, Palo Alto, CA (US)

(21) Appl. No.: 17/169,331

(22) Filed: Feb. 5, 2021

Related U.S. Application Data
(60) Provisional application No. 62/971,107, filed on Feb. 6, 2020.

(51) Int. Cl. G01B 17/02

(52) U.S. Cl. CPC G01B 17/025

(57) ABSTRACT

A system for detecting the presence of biofilm on an inner surface of a body used for containing a fluid medium. The system includes a transmitter disposed at a first location external to the body, a receiver located at a second location external to the body, and an electronic controller. The electronic controller configured to control the transmitter to transmit an ultrasonic signal in a direction towards the body and receive, via the receiver, an attenuated signal that is the ultrasonic signal after passing through the body. The electronic controller is configured to determine a phase shift between the ultrasonic signal and the attenuated ultrasonic signal, determine an amplitude difference between the ultrasonic signal and the attenuated ultrasonic signal, and generate an indication of an amount of biofilm present on the inner surface of the body based on the phase shift and the amplitude difference.

### RELATED APPLICATIONS

This application claims the benefit of U.S. Provisional Patent Application No. 62/971,107, filed Feb. 6, 2020, the entire content of which is hereby incorporated by reference.

### STATEMENT REGARDING FEDERALLY SPONSORED RESEARCH

This invention was made with government support under 1540032 awarded by the National Science Foundation. The government has certain rights to this invention.

### FIELD

Embodiments described herein relate to systems and methods for detecting the presence of biofilm.

### SUMMARY

Biofilm is, for example, a matrix-enclosed accumulation of dense microbial consortia that lives on a biological or engineering surface. Biofilm may form when single cells and microcolonies of bacteria attach and adhere to a surface over time. In many instances, biofilm forms on surfaces that come into contact with substances intended for human exposure (e.g., through consumption or external contact), such as food and water. In such instances, exposure to a substance that comes into contact with biofilm may result in illness.

Unfortuneately, many surfaces on which biofilm forms are not readily visible. For example, biofilm may form on the inner surfaces of pipes and tanks that contain substances intended for human exposure. Incorporating sensors inside pipes or tanks can be problematic, as maintenance on the sensors cannot be performed without dissessmbly of the system to be measured and the sensors themselves may be exposed to an environment that degrades the sensors over time. Accordingly, exisitng systems and methods used for detecting the formation of biofilm on such inner surfaces are ineffective.

In one embodiment, a system is provided detecting the presence of biofilm on an inner surface of a body used for containing a fluid medium. The system includes a transmitter disposed at a first location external to the body, a receiver located at a second location external to the body, and an electronic controller. The electronic controller is configured to control the transmitter to transmit an ultrasonic signal in a direction towards the body and receive, via the receiver, an attenuated signal that is the ultrasonic signal after passing through the body. The electronic controller is configured to determine a phase shift between the ultrasonic signal and the attenuated ultrasonic signal, determine an amplitude difference between the ultrasonic signal and the attenuated ultrasonic signal, and generate an indication of an amount of biofilm present on the inner surface of the body based on the phase shift and the amplitude difference.

In another embodiment, a method is provided for detecting the presence of biofilm on an inner surface of a body used for containing a fluid medium. The method includes controlling, by an electronic processor, a transmitter disposed at a first location external to the body to transmit an ultrasonic signal in a direction towards the transmitter and receiving, by the electronic controller, via a receiver disposed at a second location external to the body, an attenuated ultrasonic signal that is the ultrasonic signal after passing through the body. The method further includes determining, by the electronic controller, a phase shift between the ultrasonic signal and the attenuated ultrasonic signal, determining, by the electronic controller, an indication of an amplitude difference between the ultrasonic signal and the attenuated ultrasonic signal, and generating, by the electronic controller, an indication of an amount of biofilm present on the inner surface of the body based on the phase shift and the amplitude difference.

Other aspects of the embodiments will become apparent by consideration of the detailed description and accompanying drawings.

### BRIEF DESCRIPTION OF THE DRAWINGS

FIG. 1 illustrates a system for detecting the presence of biofilm, according to some embodiments.

FIG. 2A illustrates a system for detecting the presence of biofilm, according to some embodiments.

FIG. 2B illustrates a system for detecting the presence of biofilm, according to some embodiments.

FIG. 3 illustrates a cutaway view of the system of FIG. 1, according to some embodiments.

FIG. 4 illustrates a cutaway view of the system of FIG. 1, according to some embodiments.

FIG. 5A illustrates a system for detecting the presence of biofilm, according to some embodiments.

FIG. 5B illustrates a system for detecting the presence of biofilm, according to some embodiments.

FIG. 5C illustrates a system for detecting the presence of biofilm, according to some embodiments.

FIG. 6 is a block diagram of a control system for the systems of FIGS. 1-5, according to some embodiments.

FIG. 7 illustrates a flow chart for detecting the presence of biofilm within the system of FIGS. 1-5, according to some embodiments.

FIG. 8A illustrates a transmitted signal in the systems of FIGS. 1-5, according to some embodiments.

FIG. 8B illustrates a received signal in the systems of FIGS. 1-5, according to some embodiments.

FIG. 9A illustrates a transmitted signal in the systems of FIGS. 1-5, according to some embodiments.

FIG. 9B illustrates a received signal in the systems of FIGS. 1-5, according to some embodiments.

FIG. 10 illustrates a system for detecting the presence of biofilm, according to some embodiments.

FIG. 11 illustrates a cutaway view of the system of FIG. 10, according to some embodiments.

FIG. 12 illustrates another cutaway view of the system of FIG. 10, according to some embodiments.

FIG. 13 illustrates a close-up view of the ultrasound signal shown in FIG. 12.

### DETAILED DESCRIPTION

Before any embodiments are explained in detail, it is to be understood that the embodiments are not limited in its application to the details of the configuration and arrangement of components set forth in the following description or illustrated in the accompanying drawings. The embodiments are capable of being practiced or of being carried out in various ways. Also, it is to be understood that the phraseology and terminology used herein are for the purpose of description and should not be regarded as limiting. The use of “including,” “comprising,” or “having” and variations thereof are meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings.

In addition, it should be understood that embodiments may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic-based aspects may be implemented in software (e.g., stored on non-transitory computer-readable medium) executable by one or more electronic processors, such as a microprocessor and/or application specific integrated circuits (“ASICs”). As such, it should be noted that a plurality of hardware and software based devices, as well as a plurality of different structural components, may be utilized to implement the embodiments. For example, “servers,” “computing devices,” “controllers,” “processors,” etc., described in the specification can include one or more electronic processors, one or more computer-readable medium modules, one or more input/output interfaces, and various connections (e.g., a system bus) connecting the components.

Relative terminology, such as, for example, “about,” “approximately,” “substantially,” etc., used in connection with a quantity or condition would be understood by those of ordinary skill to be inclusive of the stated value and has the meaning dictated by the context (e.g., the term includes at least the degree of error associated with the measurement accuracy, tolerances [e.g., manufacturing, assembly, use, etc.] associated with the particular value, etc.). Such terminology should also be considered as disclosing the range defined by the absolute values of the two endpoints. For example, the expression “from about 2 to about 4” also discloses the range “from 2 to 4.” The relative terminology may refer to plus or minus a percentage (e.g., 1%, 5%, 10%, or more) of an indicated value.

Functionality described herein as being performed by one component may be performed by multiple components in a distributed manner. Likewise, functionality performed by multiple components may be consolidated and performed by a single component. Similarly, a component described as performing particular functionality may also perform additional functionality not described herein. For example, a device or structure that is “configured” in a certain way is configured in at least that way but may also be configured in ways that are not explicitly listed.

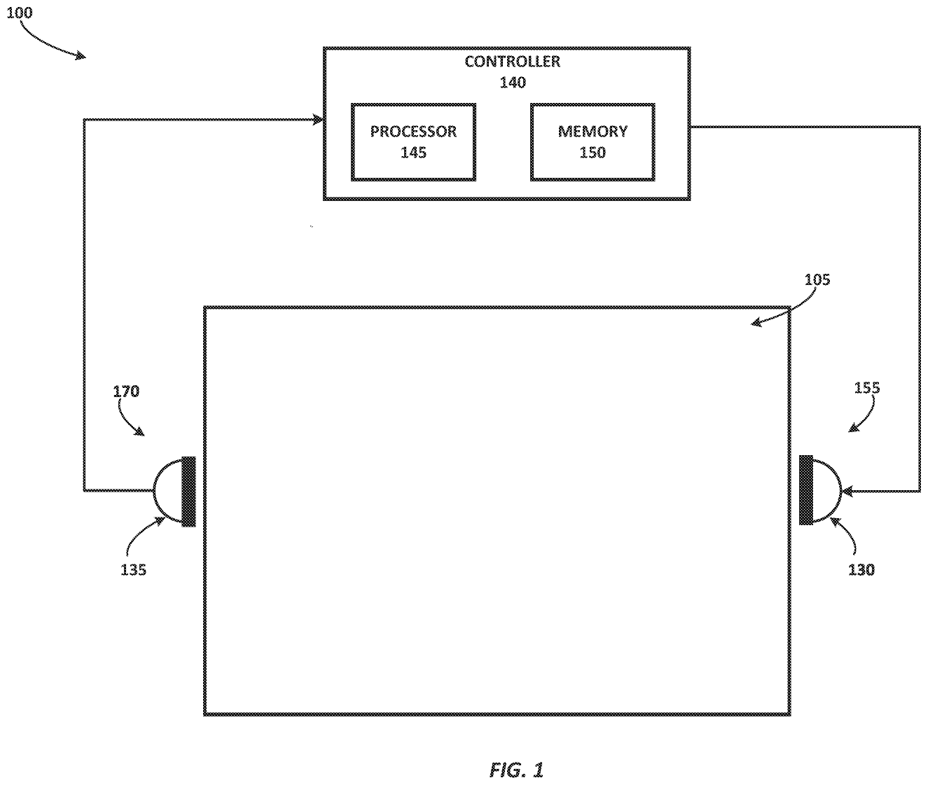

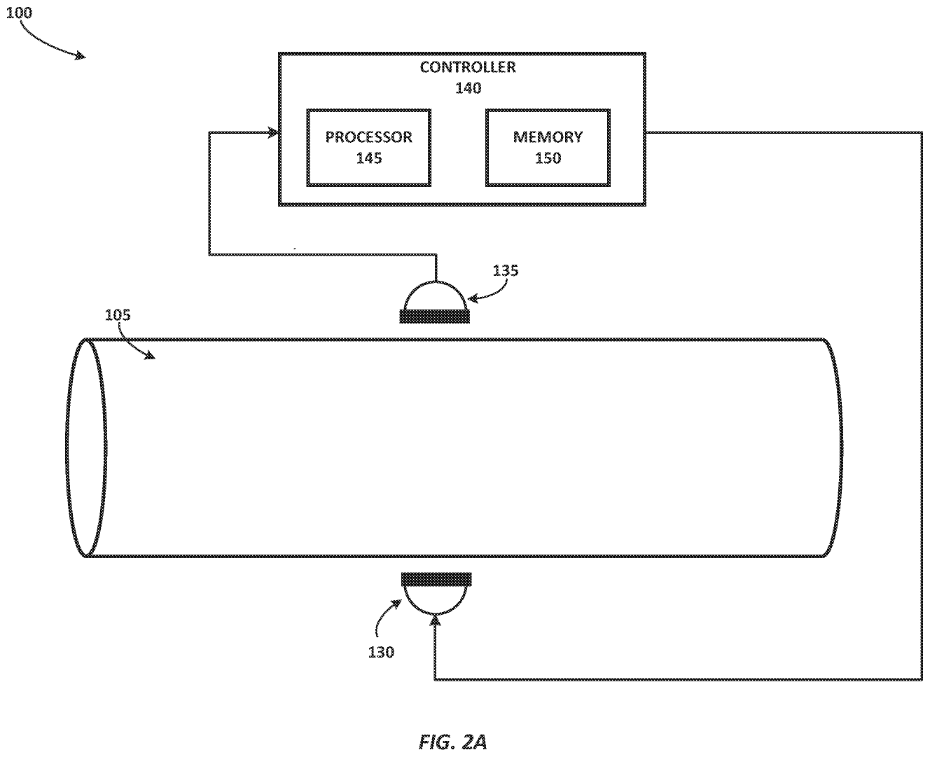

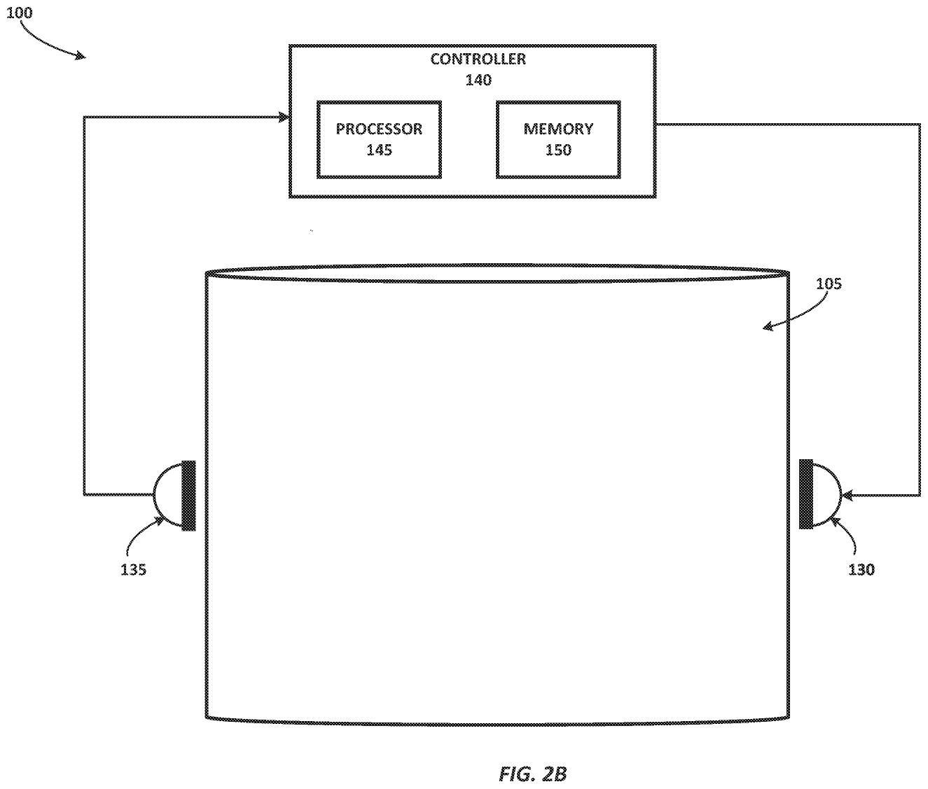

FIG. 1 illustrates a schematic view of a biofilm detection system 100 for detecting the presence of biofilm on an inner surface of a body 105. The body 105 may be implemented as any type of body that is used for containing a fluid medium. For example, the fluid medium contained in body 105 may be water, air, chemicals, medicine, blood, beverages, food, other substances intended for human exposure, or a combination thereof. In some embodiments, the body 105 is implemented as, for example, a pipe, a tank, a flexible tube, a flexible container, or another type of vessel used for containing a fluid medium. For example, FIG. 2A illustrates an embodiment of the system 100 in which the body 105 is a pipe. As another example, FIG. 2B. illustrates an embodiment of the system 100 in which the body 105 is a tank.

In some embodiments, the body 105 is included as one or more components of a larger system. For example, in some embodiments, the body 105 is implemented as one or more components of an appliance such as an ice machine, a coffee maker, a refrigerator, a freezer, or another machine used for processing foods and beverages intended for human consumption. In some embodiments, the body 105 is implemented as one or more components included in a food storage system, a water filtration system, a plumbing system, a sewage system, or a heating, ventilation, and/or air conditioning (HVAC) system. In some embodiments, the body 105 is implemented as one or more components of a medical device such as an air purifier, a ventilator, a dialysis machine, equipment used for blood transfusion, or another non-invasive, invasive, or active medical device.

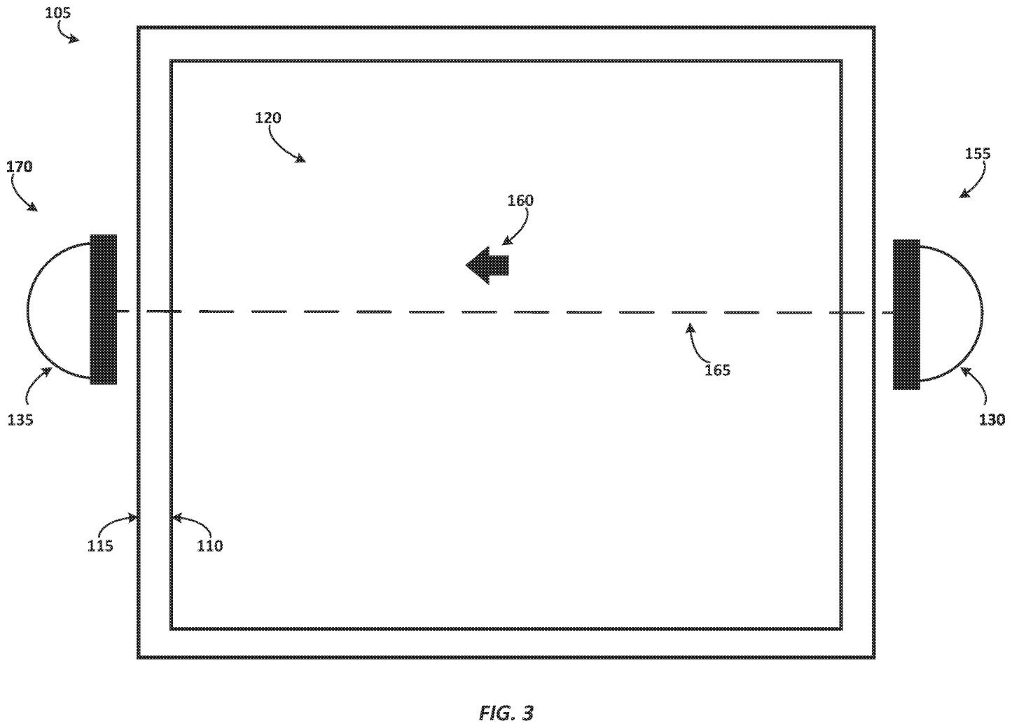

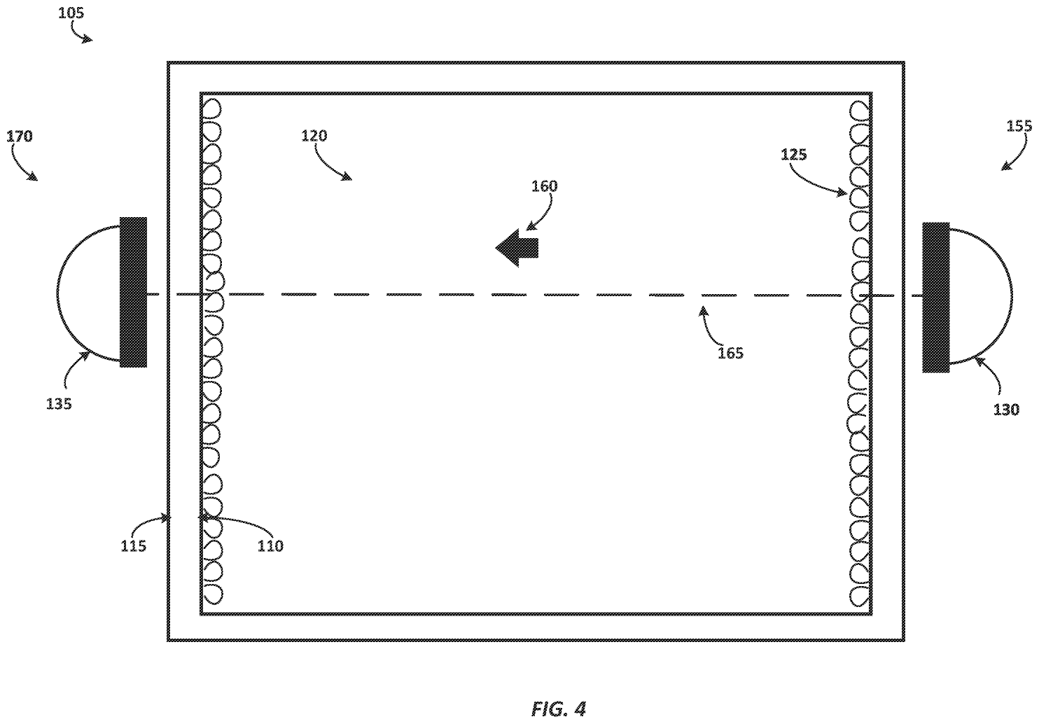

FIGS. 3-4 illustrate a close-up view of the system 100 in which a cutaway, or cross-section, of the body 105 is shown. Although the body 105 illustrated in FIGS. 3-4 is generally box-shaped, it should be understood that description herein is also applicable to embodiments in which the body 105 is not box-shaped, such as when body 105 is a cylindrical pipe or tank. The body 105 includes an inner surface 110 and an outer surface 115. The inner surface 110 defines at least in part the body's interior 120 in which the fluid medium is contained. In some embodiments, the body 105 may be composed of an opaque material such that the outer surface 115 blocks visibility of the inner surface 110 and interior 120. Accordingly, in such embodiments, formation of biofilm 125 on the inner surface 110 of the body 105 is not visible=or easily detected from a perspective that is outside of the body 105 (FIG. 3). For example, the body 105 may be composed of materials such as, but not limited to, polyvinyl chloride (PVC), acrylonitrile butadiene styrene (ABS), chlorinated polyvinyl chloride (CPVC), high-density polyethylene (HDPE), cross-linked polyethylene (PEX), copper, steel, galvanized iron, brass, carbon steel, and/or any other types of polymers or metals.

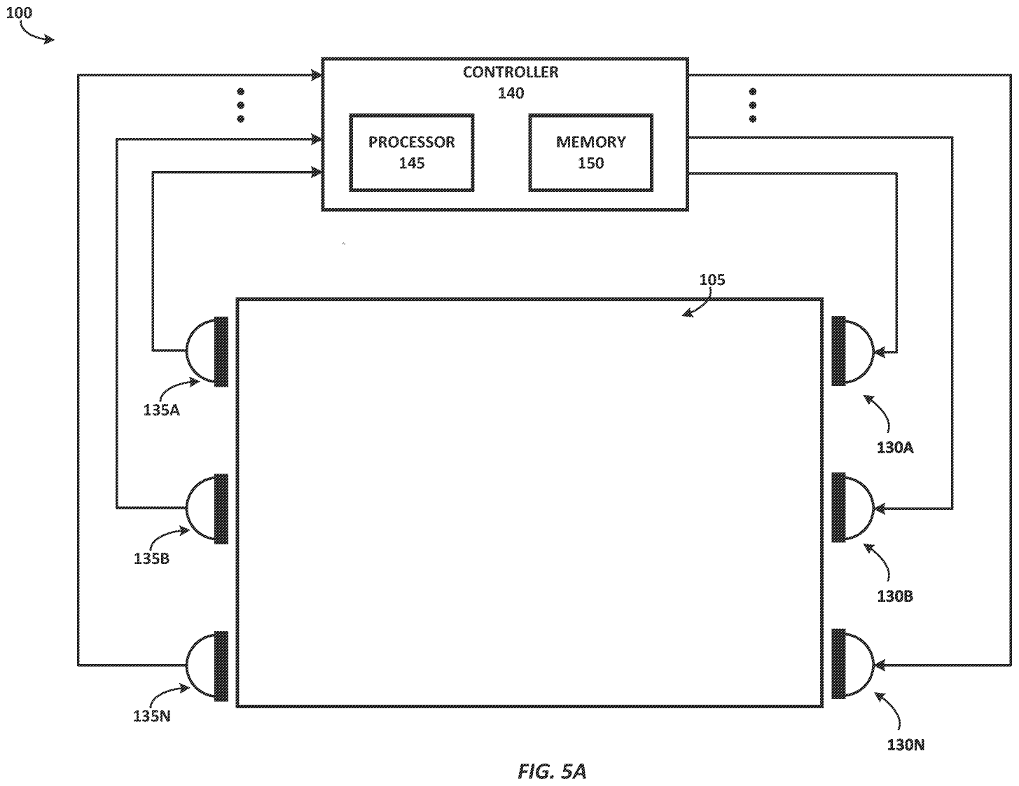

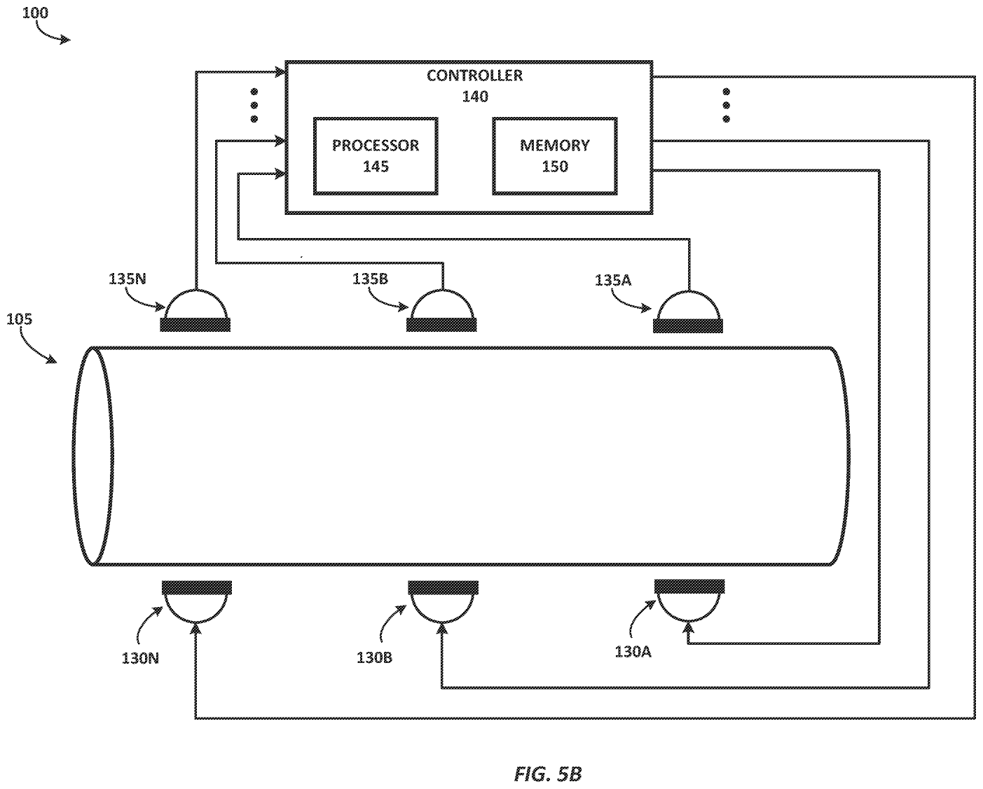

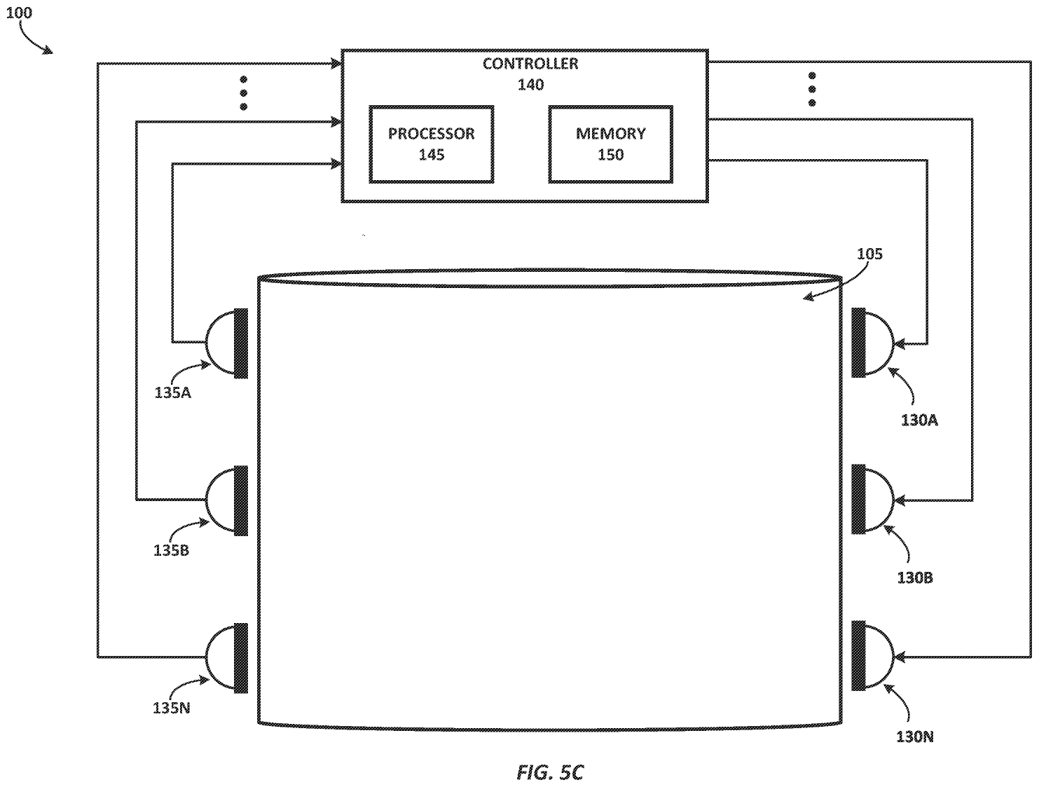

As shown in FIGS. 1-4, the system 100 further includes at least one transmitter 130 and at least one receiver 135. Although the system 100 is shown as including only one transmitter 130 and one receiver 135 in FIGS. 1-4, it should be understood that system 100 may include any number of transmitters and receivers that are desired. For example, FIGS. 5A-5C illustrate embodiments of the system 100 in which the system 100 includes a plurality of transmitters 130A-130N and a plurality of receivers 135A-135N. The description of the transmitter 130 and the receiver 135 herein generally applies to the plurality of transmitters 130A-130N and the plurality of receivers 135A-135N, respectively.

In some embodiments, the transmitter 130 and receiver 135 are implemented as ultrasonic sensors. That is, the transmitter 130 is an ultrasonic transmitter and the receiver 135 is an ultrasonic receiver. In such embodiments, the transmitter is configured to transmit ultrasonic signals and the receiver 135 is configured to receive ultrasonic signals. Operation of the transmitter 130 and receiver 135 is controlled by an electronic controller 140 (see FIGS. 1, 2A, 2B, and 5A-5C) which is operatively coupled to the transmitter 130 and receiver 135. As will be described in more detail below, the electronic controller 140 includes an electronic processor 145 and a memory 150.

As shown in FIGS. 1 and 3-4, the transmitter 130 is located at a first position 155 external to the body 105. In the illustrated embodiment, the first position 155 at which transmitter 130 is located is external to the right side of body 105. However, it should be understood that the position 155 at which transmitter 130 is located, as shown in FIGS. 1 and 3-4, is provided by way of example only. Moreover, persons skilled in the art will further understand that the transmitter 130 may be located at various other positions external to the body 105. For example, in some embodiments, the transmitter 130 may be located at a position that is external to the left side of body 105, a position that is external to the top side of body 105, a position that is external to the bottom side of body 105, a position that is external to the front side of the body 105, a position that is external to the rear side of the body 105, or another position that is external to the body 105.

While located at the first position 155 external to body 105, the transmitter 130 may be oriented such that when the transmitter 130 transmits an ultrasonic signal, the ultrasonic signal is transmitted in a direction (indicated by arrow 160 in FIGS. 3-4) towards the body 105. For example, FIGS. 3 and 4 illustrate an exemplary ultrasonic signal 165 that is transmitted by the transmitter 130 in a direction towards the body 105. The ultrasonic signal 165 may pass through the body 105 such that the ultrasonic signal 165 enters through the outer surface 115 of the right side of body 105, passes through the interior 120, and exits through the outer surface 115 of the left side of the body 105. In some embodiments, the transmitter 130 is oriented such that ultrasonic signals transmitted by the transmitter 130 intersect the outer surface 115 of the right side of body 105 at an approximately right angle (e.g., 90 degrees, between 85-95 degrees, between 80-100 degrees). In some embodiments, ultrasonic signals transmitted by transmitter 130 intersect the outer surface 115 of body 105 at an offset angle (e.g., offset by 1-45 degrees with respect to the perpendicular).

With respect to the embodiments in which the system 100 includes a plurality of transmitters 130A-130N and receivers 135A-135N, the plurality of transmitters 130A-130N may be located at various positions external to the body 105. As shown in FIG. 5A, the plurality of transmitters 130A-130N are located in various positions external to the right side of body 105. However, it should be understood that the plurality of transmitters 130A-130N are not limited in location to positions external to the right sight of the body 105. For example, in some embodiments, the plurality of transmitters 130A-130N may located at positions external to the left side of body 105, positions external to the top side of body 105, positions external to the bottom side of body 105, positions external to the front side of body 105, positions external to the rear side of body 105, or other positions external to the body 105. Furthermore, it should be understood that each one of the plurality of transmitters 130A-130N need not be located at positions external to the same side of body 105. For example, in some embodiments, a first transmitter 130A may be located at a position external to the right side of body 105 and a second transmitter 130B may be located at a position external to the top side of body 105. In some embodiments, the locations of the plurality of transmitters 130A-130N are chosen in accordance with a geometry of the body. For example, as shown in the embodiment of FIG. 5B in which the body 105 is a pipe, the plurality of transmitters 130A-130B may be located at positions distributed along the length of the exterior of the pipe. As another example, as shown in the embodiment of FIG. 5C in which the body 105 is a tank, the plurality of transmitters 130A-130B may be located at positions distributed along the exterior of the tank. The above described locations of the plurality of transmitters 130A-130N are provided by way of example and do not limit the plurality of transmitters 130A-130N from being located at positions external to the body 105 not explicitly disclosed above.

As further shown in FIGS. 1 and 3-4, the receiver 135 is located at a second position 170 external to the body 105. In the illustrated embodiment, the second position 170 at which the receiver 135 is located is external to the left side of body 105. Thus, relative to the body 105, the receiver 135 is located at a position opposite to the position at which the transmitter 130 is located. However, it should be understood that the position 170 at which the receiver 135 is located, as shown in FIGS. 1-3, is provided by way of example. The receiver 135 may be located at various other positions external to the body 105. In some embodiments, the receiver 135 may be located at a position that is external to the right side of body 105, a position that is external to the top side of body 105, a position that is external to the bottom side of body 105, a position that is external to the front side of the body 105, a position that is external to the rear side of the body 105, or another position that is external to the body 105.

In some embodiments, the location of receiver 135 is chosen in accordance with the location of the transmitter 130. In such embodiments, the location of receiver 135 may be chosen to be a position external to body 105 at which the receiver 135 is positioned to receive the ultrasonic signal 165 that is transmitted by the transmitter 130. For example, while the receiver 135 is located at the second position 170, the receiver 135 is operable to receive the ultrasonic signal 165 when the ultrasonic signal 165 passes through the body 105, and without primarily relying on internal reflection of the ultrasonic signal 165 within the body 105. As another example, when the transmitter 130 is located in a position external to the top side of the body 105, the location of the receiver 135 may be chosen to be a position external to the bottom side of the body 105. Accordingly, while located at a position external to the bottom side of the body 105, the receiver 135 is operable to receive an ultrasonic signal passing through body 105 that is transmitted by a transmitter 130 located at a position external to the top side of body 105. As another example, when the transmitter 130 is located in a position external to the left side of the body 105, the location of the receiver 135 may be chosen to be a position external to the right side of the body 105. Accordingly, while located at a position external to the right side of the body 105, the receiver 135 is operable to receive an ultrasonic signal passing through body 105 that is transmitted by a transmitter 130 located at a position external to the left side of body 105. In general, the location of the receiver 135 may be chosen to be a position external to a side or area of the body 105 that is opposite to the side or area external to the body 105 at which transmitter 130 is located. In general, when the transmitter 130 and the receiver 135 are located on opposite sides of the body 105 (as illustrated in FIGS. 1-6), the receiver is configured to receive the ultrasonic signal 165 emitted from the transmitter 130 after the signal passes through the body 105 and without an internal reflection of the ultrasonic signal 165 within the body 105. That is, although some internal reflection of the ultrasonic signal 165 may occur and be detected by the receiver 135, the transmitter 130 and the receiver 135 are positioned such that the ultrasonic signal 165 received by the receiver 135 travels in a generally straight path from the transmitter 130 to the receiver 135 without internal reflection within the body 105. Such alignment allows for a stronger signal to be received and detected by the receiver 135.

With respect to the embodiments in which the system 100 includes a plurality of transmitters 130A-130N and receivers 135A-135N, the plurality of receivers 135A-135N may be located at various positions external to the body 105. As shown in FIG. 5A, the plurality of receivers 135A-135N are located in various positions external to the left side of body 105. While located at the various positions external to the left side of body 105, the each of the plurality of receivers 135A-135N is operable to receive an ultrasonic signal transmitted by a respective one of the plurality of transmitters 130A-130N located at various positions external to the right side of the body 105. However, it should be understood that the plurality of receivers 135A-135N are not limited in location to positions external to the right sight of the body 105. For example, in some embodiments, the plurality of receivers 135A-135N may be located at positions external to the left side of body 105, positions external to the top side of body 105, positions external to the bottom side of body 105, positions external to the front side of body 105, or other positions external to the rear side of body 105.

Similar to the single transmitter 130 and single receiver 135 embodiment described above, the locations of receivers 135A-135N may be chosen in accordance with the locations of the transmitters 130A-130N. In such embodiments, the location of each one of the plurality of receivers 135A-135N may be chosen to be a position external to the body at which the respective one of the plurality of receivers 135A-135N is operable to receive an ultrasonic signal transmitted by a respective one of the plurality of transmitters 130A-130N. For example, with respect to FIG. 5A, the receiver 135A is located at a position external to the left side of body 105 such that the receiver 135A is operable to receive ultrasonic signals passing through the body 105 that are transmitted by the transmitter 130A, which is located in a position external to the right side of the body 105. In some embodiments, respective ones of the plurality of receivers 135A-135N may be located at positions external to varying sides of the body 105. For example, when the transmitter 130A is located in a position external to the right side of body 105, the location of receiver 135A may be chosen to be a position external to the left side of body 105. Similarly, when the transmitter 130B is located in a position external to the top side of body 105, the location of receiver 135B may be chosen to be a position external to the bottom side of body 105. Likewise, when the transmitter 130N is located in a position external to the front side of body 105, the location of receiver 135N may be chosen to be a position external to the rear side of body 105. In general, the location of a particular one of the plurality of transmitters 135A-135N may be chosen to be a position external to a side or area of the body 105 that is opposite to the side or area external to the body 105 at which a respective one of the plurality of transmitters 130A-130N is located. Accordingly, for every one of the plurality of transmitters 130A-130B that transmits an ultrasonic signal in a direction towards the body 105, there is at least one receiver 135 located in a position external to the body 105 such that the at least one receiver 135 is operable to receive the ultrasonic signal when it passes through the body 105.

As described above, operation of the transmitters 130 and receivers 135 is controlled by the electronic controller 140. FIG. 6 illustrates a block diagram of a control system 600 of the system 100 according to some embodiments. The control system 600 includes the electronic controller 140, the one or more transmitters 130, and the one or more receivers 135 of the system 100. The electronic controller 140 is electrically and/or communicatively connected to a variety of modules or components of the control system 600. For example, the controller 140 is connected to the one or more transmitters 130, the one or more receivers 135, a power supply 605, one or more sensors 610, a user-interface 615, and a communication circuit 620.

The power supply 605 is configured to provide power to the electronic controller 140 and/or other components of the control system 600. In some embodiments, the power supply 605 includes a battery pack configured to provide power to the electronic controller 140 and/or other components of the control system 600. In some embodiments, the power supply 605 may receive power directly from an external power source, such as an AC wall outlet or a generator, and provide the received power to the electronic controller 140 and/or other components of the control system 600. The power supply may include DC-DC converters, AC-DC converters, DC-AC converters, and/or AC-AC converters configured for regulating the power provided to the electronic controller 140 and/or other components of the control system 600.

The one or more sensors 610 are configured to sense one or more characteristics of the system 100. In some embodiments, the sensors 610 include one or more voltage sensors and current sensors for sensing respective voltages and currents of components of the system 100. For example, in some embodiments, a voltage sensor is provided to sense respective voltages of the signals driving the transmitter 130 to generate ultrasonic signals and the signals generated by the receiver 135 in response to received ultrasonic signals. In some embodiments, the sensors 610 may include one or more temperature sensors configured to sense temperatures within and/or nearby the system 100. In some embodiments, one or more of the sensors 610 are incorporated into the controller 140.

The user-interface 615 may be configured to receive input from an operator (e.g., a service technician or other user) of the system 100. The user-interface 615 may additionally be configured to output information concerning a status of the system 100 to an operator and/or owner of the system 100. In some embodiments, the user-interface 615 includes a display (for example, a primary display, a secondary display, etc.) for providing visual feedback to an operator and/or input devices (for example, touchscreen displays, a plurality of knobs, dials, switches, buttons, etc.). The display may be, for example, a liquid crystal display (“LCD”), a light-transmitting diode (“LED”) display, an organic LED (“OLED”) display, an electroluminescent display (“ELD”), a surface-conduction electron-transmitter display (“SED”), a field emission display (“FED”), a thin-film transistor (“TFT”) LCD, etc. In some embodiments, the user-interface 615 includes one or more speakers for providing audible feedback to an operator of the system 100.

The communication circuit 620 is configured to enable communication between the electronic controller 140 and one or more external devices (for example, smart phones, tablets, laptops, computers, servers, etc.). The one or more external devices may be owned and/or operated by owners of the system 100, operators of the system 100, a service utility provider, maintenance personnel, healthcare workers, manufacturing workers, and the like. The communication circuit 620 may include one or more antennas, communication ports, wireless transmitters, wireless receivers, and/or wireless transceivers. The communication circuit 620 may be configured to communicate with the one or more external devices using a wired and/or wireless connection. For example, in some embodiments, the communication circuit 620 may be configured to communicate with the one or more external devices using short-range radio communication (e.g., Bluetooth®, WiFi®, NFC, ZigBee®, etc.). In some embodiments, the communication circuit 620 may be configured to communicate with the one or more external devices using long-range radio communication (e.g., cellular communication over a cellular network). In some embodiments, the communication circuit 620 may be configured to communicate with one or more external devices using a wired connection.

As described above, the electronic controller 140 may include a plurality of electrical and electronic components that provide power, operational control, and protection to the components and modules within the control system 600. For example, the electronic controller 140 includes, among other things, the electronic processor 145 (for example, a microprocessor or another suitable programmable device) and the memory 150. In some embodiments, the electronic controller 140 may be implemented using an Arduino®, Raspberry Pi®, or similar controller. In some embodiments, the electronic controller 140 may include or incorporate similar functionality provided by a time-to-digital converter evaluation microchip, such as the Texas Instruments Time to Digital Converter 1000-7200 EVM Board®. In some embodiments, the electronic controller 140 includes one or more of current sensing, voltage sensing, and frequency sensing, and time sensing capabilities (e.g., through hardware circuit components, software, or a combination thereof) to measure current and voltage amplitudes, current and voltage zero-crossings, elapsed times, and phase shifts.

The memory 150 may include, for example, a program storage area and a data storage area. The program storage area and the data storage area can include combinations of different types of memory, such as read-only memory (ROM) and random access memory (RAM). Various non-transitory computer readable media, for example, magnetic, optical, physical, or electronic memory may be used. The electronic processor 145 is communicatively coupled to the memory 150 and executes software instructions that are stored in the memory 150, or stored in another non-transitory computer readable medium such as another memory or a disc. The software may include one or more applications, program data, filters, rules, one or more program modules, and other executable instructions. For example, in some embodiments, the electronic processor 145 is configured to retrieve and execute instructions from the memory 150 to implement the functionality of the controller 140 described herein.

As noted, the electronic controller 140 is configured to control the one or more transmitters 130 and the one or more receivers 135 of the system 100. For example, the electronic controller 140 is configured to drive each of the one or more transmitters 130 to cause the output of respective ultrasonic signals from each transmitter 130. For example, the electronic controller 140 may provide a command (e.g., one or more digital or analog signals) to each transmitter 130 that specifies the output characteristics of the ultrasonic signal (e.g., the amplitude and frequency) to be output. Each transmitter 130, which may be powered via the controller 140 or directly via the power supply 605, then generates and outputs the requested ultrasonic signal. In other words, each transmitter 130 receives one or more signals from the controller 140 and, in response, generates a respective ultrasonic signal. In some embodiments, the electronic controller 140 drives each transmitter 130 with an analog sine wave (or another periodic wave), which the transmitters 130 then convert and output as respective ultrasonic signals. Additionally, each of the one or more receivers 135 is configured to receive an ultrasonic signal and provide the received ultrasonic signals (e.g., in an analog or digital form) to the electronic controller 140. In some embodiments, the one or more receivers 135 each receive an ultrasonic signal, which each receiver 135 then respectively converts and outputs to the electronic controller 140 as respective analog sine waves (or as other periodic waves). In some embodiments, the transmitter 130 is an ultrasound sensor similar to an ultrasound sensor sold by Osenon Technology with part number 1ME21TR-1, or similar to an ultrasonic sensor sold by various sources under part number HC-SR04.

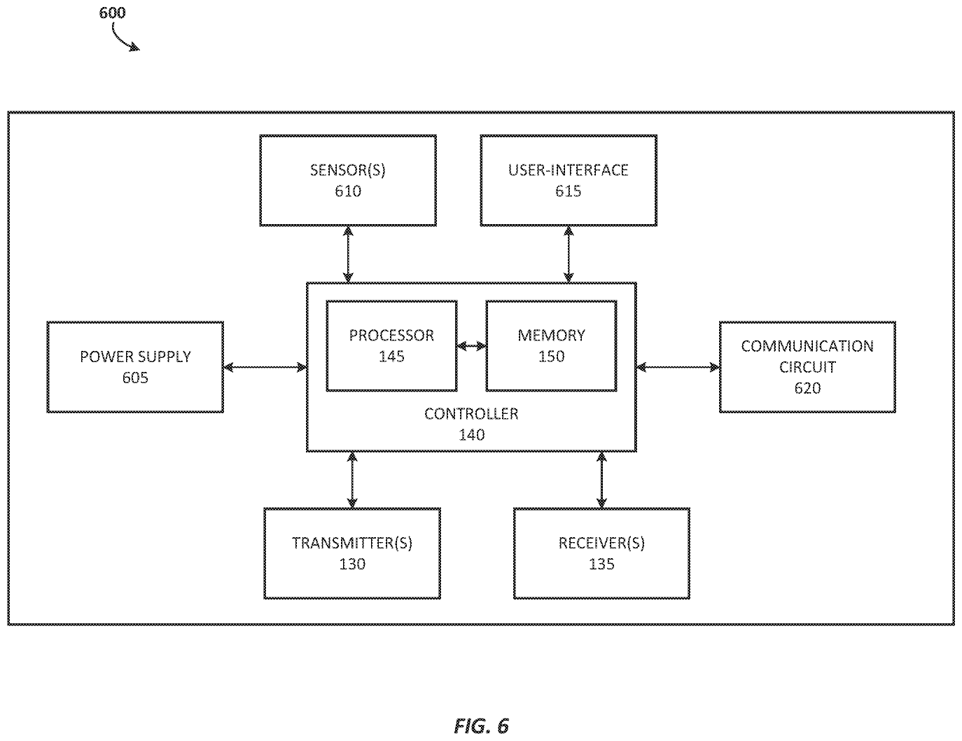

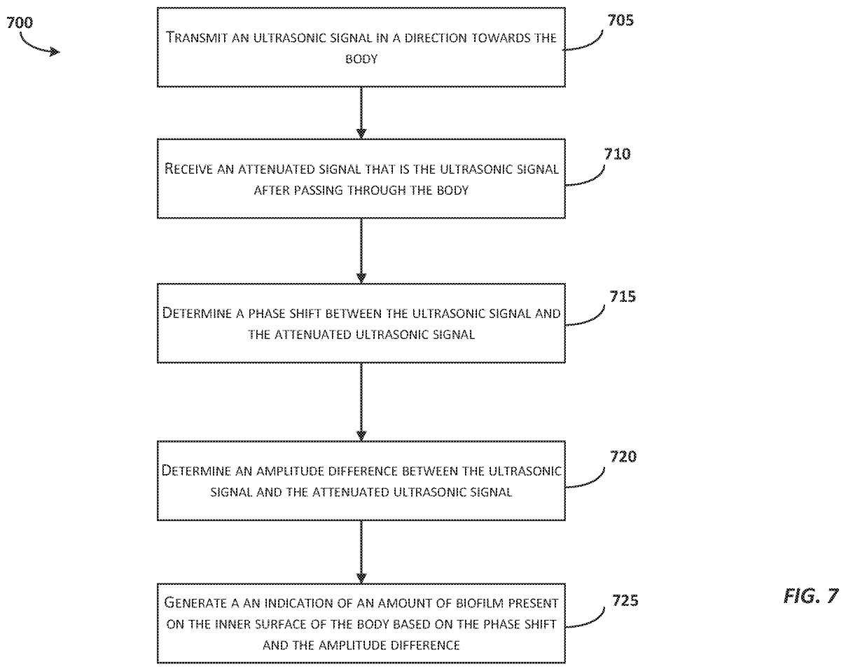

FIG. 7 illustrates a method 700 for detecting the presence of biofilm on an inner surface of a body used for containing a fluid medium and includes blocks 705, 710, 715, 720, and 725. For the sake of explanation, the method 700 is primarily described with respect to the embodiment of detection system 100 that includes the transmitter 130, the receiver 135, and the electronic controller 140 and that detects biofilm with respect to the body 105. However, it should be understood that the method 700 as described herein may also be used to detect the presence of biofilm in embodiments of system 100 that include the plurality of transmitters 130A-130N, the plurality of receivers 135A-135N, and the electronic controller 140. Furthermore, the method 700 is described generally with respect to detecting the presence of biofilm on an inner surface of the body 105, which should be understood to include the various types of bodies described above (e.g., a pipe, a tank, and/or any other body used for containing a fluid medium). Additionally, although actions within the method 700 are described as being carried out by the electronic controller 140, the actions may also be carried out by other electronic controllers or systems.

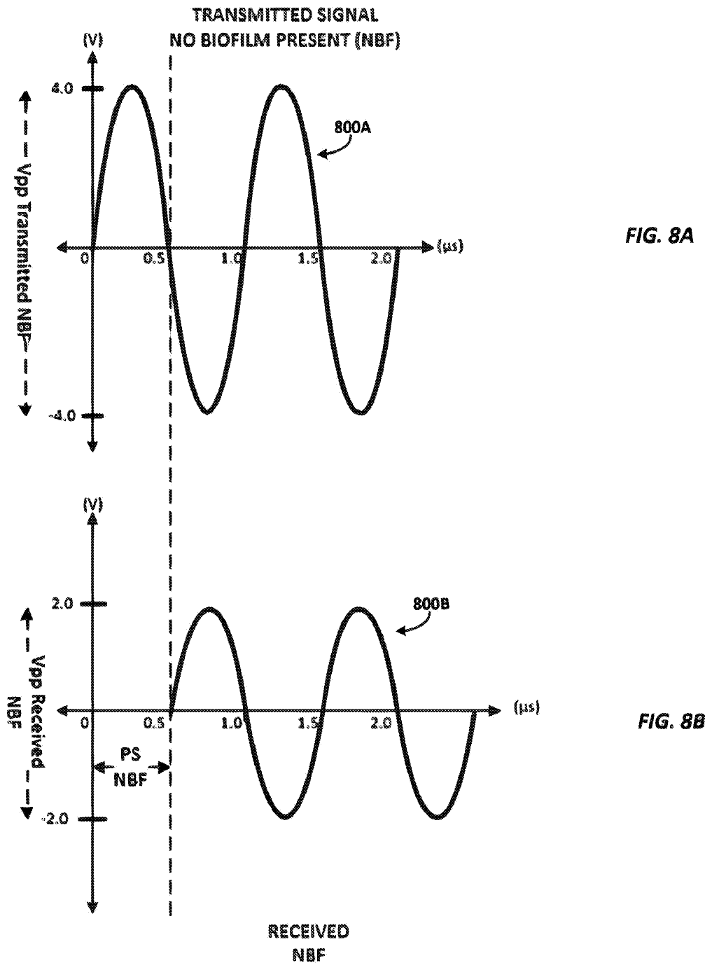

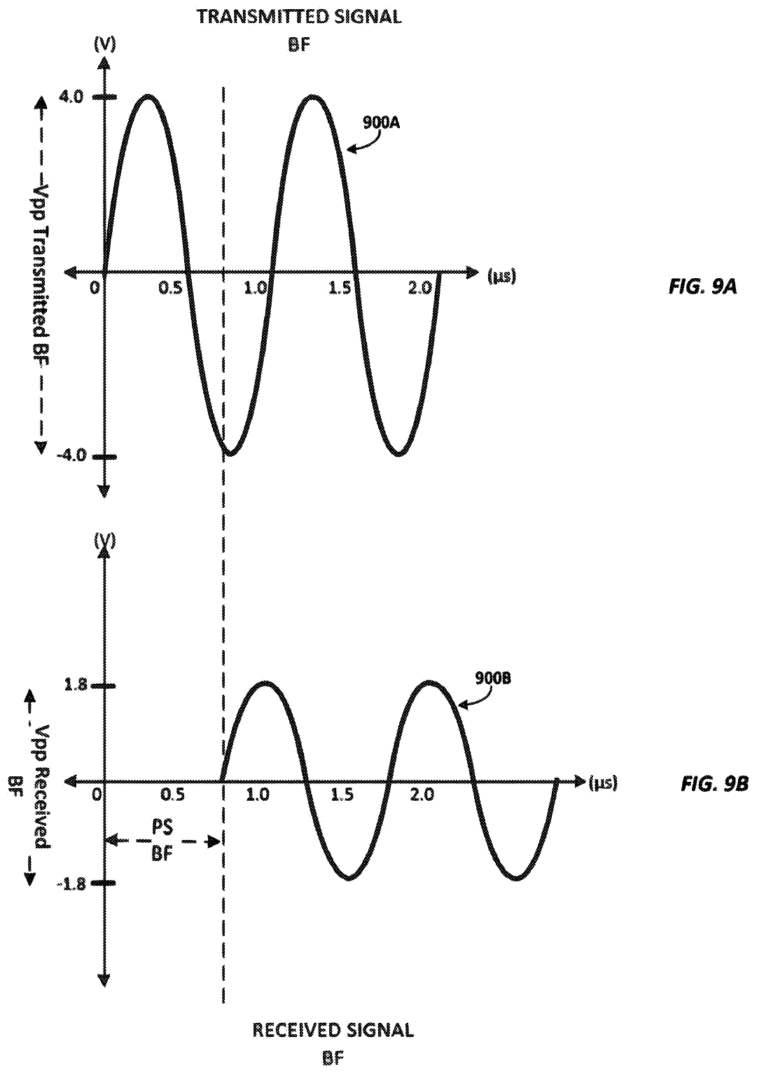

In block 705, the electronic controller 140 controls the transmitter 130 to transmit an ultrasonic signal in a direction towards the body 105. For example, the electronic controller 140 may provide one or more signals to the transmitter 130 (e.g., a signal in the form of an analog sine wave) that drives the transmitter 130 to output the ultrasonic signal. FIGS. 8A and 9A illustrate exemplary ultrasonic signals transmitted by the transmitter 130 in a direction towards the body 105. More particularly, FIG. 8A illustrates a representation of an ultrasonic signal that is transmitted by the transmitter 130 in a direction towards the body 105, hereinafter referred to as the transmitted signal 800A, and FIG. 9A illustrates an ultrasonic signal 900A that is transmitted by the transmitter 130 in a direction towards the body 105, hereinafter referred to as the transmitted signal 900A. In some embodiments, the transmitted signals 800A and 900A are analog electrical signals that drive the transmitter 130 to generate the respective ultrasonic signals. These analog electrical signals may be measured by the electronic controller 140 (e.g., via a voltage sensor of the sensors 610) or may be presumed (without separate measurement) by the electronic controller 140 because the electronic controller 140 is generating these analog electrical signals. Because these transmitted signals 800A and 900A cause the transmitter 130 to generate ultrasonic signals and serve as a proxy for the generated ultrasonic signals, and to simplify the explanation of the system and method herein, the transmitted signals 800A and 900A may be referred to as ultrasonic signals (unless otherwise specified).

The transmitted signal 800A is transmitted in a scenario in which there is no biofilm present on an inner surface 110 of the body 105. The transmitted signal 900A is transmitted in a scenario in which biofilm is present on an inner surface 110 of the body 105. It will be assumed that the body 105 through which transmitted signal 800A passes is the same body 105 through which transmitted signal 900A passes. That is, the body 105 through which transmitted signal 800A passes is composed of the same thickness and type of material through which transmitted signal 900A passes. The difference between transmitted signals 800A and 900A is that transmitted signal 800A passes through the body at a time in which no biofilm is present on an inner surface 110 of body 105 (e.g., the time at which body 105 has been manufactured, installed, and/or recently cleaned) and transmitted signal 900A passes through the body at a time in which biofilm is present on an inner surface 110 of the body 105 (e.g., some time after the body 105 has been manufactured, installed, and/or cleaned). Since the transmitted signals 800A and 900A are equivalent, the below description provided with respect to the transmitted signal 800A is similarly applicable to the transmitted signal 900A.

As shown in FIG. 8A, the transmitted signal 800A is a sinusoidal signal having an amplitude of 8 volts peak-to-peak (Vₚₚ) and a frequency of 1 megahertz (MHz). It should be understood that the amplitude and frequency of the transmitted signal 800A are provided by way of example and do not limit the transmitted signal 800A from having amplitude and/or frequency values that are different from the values shown in FIG. 8A. Furthermore, although illustrated as being a sinusoidal signal, it should be understood that the transmitted signal 800A may alternatively be a periodic signal having rectangular, sawtooth, triangular, or another shaped wave.

The amplitude and/or frequency of the transmitted signal 800A may be selected and generated based on the type of material from which body 105 is constructed. For example, the electronic controller 140 may receive, from an operator (via the user-interface or via an external device and the communication circuit 620), body characteristics indicating the type, thickness, or both the type and thickness of the material of the body. In turn, the electronic controller 140 may be configured to select the amplitude, frequency, or both the amplitude and frequency of the transmitted signal 800A based on the type of material from which body 105 is formed. For example, the body characteristics may be used as input to a lookup table maintained on the electronic controller 140 (e.g., in the memory 150), and the amplitude and frequency for the transmitted signal 800A may be provided as an output by the lookup table. Among other things, the type and/or thickness of a material from which body 105 is formed effects the extent to which the transmitted signal 800A becomes attenuated as the transmitted signal 800A passes through the body 105. For example, ultrasonic signals that pass through materials such as copper or galvanized steel may be attenuated, or reduced in strength, by a greater amount than when compared to the attenuation of an ultrasonic signal that passes through materials such as PVC. As another example, ultrasonic signals that pass through a material that is three inches thick may be attenuated, or reduced in strength, by a greater amount than when compared to the attenuation of an ultrasonic signal passes through a one inch thick sample of the same material. Similarly, the phase shift experienced by the transmitted signal 800A as it passes through body 105 may be affected by the type and/or thickness of the material from which body 105 is formed.

In some embodiments, the amplitude and/or frequency of the transmitted signal 800A may be selected so as to (i) prevent the ultrasonic signal from being diminished while passing through and exiting the body 105 so much that it may not be adequately detected by the receiver 135 and (ii) so that the resulting phase shift experienced by the transmitted signal 800A will be sufficiently large to be able to be adequately detected by the receiver 135. That is, the amplitude of the transmitted signal 800A may be chosen to be a value that is large enough (e.g., 5 Vₚₚ) such that when the transmitted signal 800A is attenuated by passing through and exiting body 105, the amplitude of the transmitted signal 800A does not decrease below a signal strength threshold (e.g., 200 mVpp) before it is received by the receiver 135. The signal strength threshold is representative of an ultrasonic signal strength at which meaningful data can no longer be obtained from the ultrasonic signal. Thus, the amplitude of transmitted signal 800A may be chosen such that the transmitted signal 800A maintains an amplitude that is greater than the signal strength threshold (e.g., 200 mVₚₚ) as it passes through body 105. For example, when the body 105 is a pipe formed of a quarter inch thick PVC, the amplitude of the transmitted signal may be chosen to be 3 Vₚₚ. As another example, when the body 105 is a tank formed of three inch thick galvanized iron, the amplitude of the transmitted signal 800A may be chosen to be 10 Vₚₚ. It should be understood that the above examples were merely provided as a means of explaining how material type may affect signal attenuation.

Selecting an amplitude for the transmitted signal 800A that is large enough to prevent the amplitude of the transmitted signal 800A from decreasing below the signal strength threshold as the transmitted signal 800A passes through and exits the body 105 may help eliminate the need for additional amplification of the transmitted signal 800A at the receiving side of body 105 (e.g., the side of body 105 at which receiver 135 is located and ultrasonic signal exits the body 105). That is, transmitting the transmitted signal with a large enough amplitude enables the receiver 135 to receive, without increasing the amplitude of the transmitted signal 800a, the transmitted signal 800A at an amplitude that is greater than the signal strength threshold. Accordingly, the size, cost, and/or power requirements associated with operating the system 100 may be reduced when compared to systems that require significant amplification of the ultrasonic signal at the receiving side.

As described above, in some embodiments, the amplitude of the transmitted signal 800A may be chosen to have any value that is large enough to prevent the amplitude of the transmitted signal 800A from being decreased below the signal strength threshold when the transmitted signal 800A passes through the body 105. However, when the amplitude of the transmitted signal 800A is chosen to have a value that is too large, the size, cost, and/or power required to operate system 100 may become undesirably large. Accordingly, in some embodiments, the amplitude of the transmitted signal 800A is less than 20 Vₚₚ, is less than 15 Vₚₚ, is less than 10 Vₚₚ, is less than 5 Vₚₚ, is between 2 and 20 Vₚₚ, is between 2 and 15 Vₚₚ, is between 2-10 Vₚₚ, or is between 2-5 Vₚₚ

Similarly, a value other than 1 MHz may be selected as the frequency of the transmitted signal 800A. However, it is preferable to choose a frequency value that is not too large because ultrasonic signals of substantially large frequencies are adversely affected by changes in temperature. When compared to the high frequency ultrasonic sensors (e.g., ultrasonic sensors operating at 15-20 MHz), medium and low frequency ultrasonic sensors are a less expensive and more reliable option. Thus, at least in some embodiments, the transmitters 130 and receivers 135 of system 100 are chosen to be ultrasonic sensors capable of operating at frequencies less than 15 MHz, less than 10 MHz, less than 5 MHz, less than 2 MHz, less than 1 Mhz, between 1 Mhz and 15 Mhz, between 1 Mhz and 10 Mhz, between 1 Mhz and 5 Mhz, between 500 Hz and 10 Mhz, or between 500 Hz and 5 Mhz.

In some embodiments, the electronic controller 140 determines the amplitude and/or frequency of the transmitted signal 800A based on a user input. For example, an operator of the system 100 (e.g., a service technician) may provide the electronic controller 140 with an input, via user-interface 615, that indicates the amplitude and/or frequency of the transmitted signal 800A. In some embodiments, the electronic controller 140 is configured to receive values for the amplitude and/or frequency of the transmitted signal 800A from an external device via the communication circuit 620. For example, an operator of the external device may transmit a signal that indicates the amplitude and/or frequency of the transmitted signal 800A to the electronic controller 140 via communication circuit 620.

Although the above description of block 705 is provided with respect to embodiments in which system 100 includes a transmitter 130 and a receiver 135, it should be understood that the above description is also applicable for embodiments of system 100 in which there are a plurality of transmitter 130A-130N and a plurality of receivers 135A-135N. For example, the electronic controller 140 may be configured to control one, some, or every one of the transmitters included in the plurality of transmitters 130A-130N to transmit a respective ultrasonic signal in a direction towards the body 105.

In block 710, the electronic controller 140 receives, via the receiver 135, an attenuated signal that is the ultrasonic signal transmitted by transmitter 130 after passing through the body 105. FIGS. 8B and 9B illustrate examples of attenuated ultrasonic signals that are received by the receiver 135 and provided to the electronic controller 140. In particular, FIG. 8B illustrates an attenuated ultrasonic signal 800B that is the attenuated transmitted signal 800A after the transmitted signal 800A passes through and exits the side of body 105 at which the receiver 135 is located. The attenuated ultrasonic signal 800B, also referred to as the received signal 800B, is received by the receiver 135 and provided to the electronic controller 140 and/or one or more other components of the control system 600. As shown in FIG. 8B, the received signal 800B is a signal received in the scenario in which there is no biofilm present on an inner surface 110 of the body 105. Similarly, FIG. 9B illustrates an attenuated ultrasonic signal 900B that is the attenuated transmitted signal 900A after the transmitted signal 900A passes through and exits the side of body 105 at which the receiver 135 is located. The attenuated ultrasonic signal 900B, also referred to as the received signal 900B, is received by the receiver 135 and provided to the electronic controller 140 and/or one or more other components of the control system 600. As shown in FIG. 9B, the received signal 900B is a signal received in the scenario in which there is no biofilm present on an inner surface 110 of the body 105. To provide the attenuated ultrasonic signal 800B and 900B to the electronic controller 140, the receiver 135 may convert the ultrasonic signal into another form, such as an electrical signal (e.g., an analog sinusoidal wave), for processing by the electronic controller 140. Unless otherwise noted, these converted ultrasonic signals are still referred to as the attenuated ultrasonic signal 800B and 900B (or as the received signals 800B and 900B).

Although the above description of block 710 is provided with respect to embodiments in which system 100 includes a transmitter 130 and a receiver 135, it should be understood that the above description is also applicable for embodiments of system 100 in which there are a plurality of transmitters 130A-130N and a plurality of receivers 135A-135N. For example, the electronic controller 140 may be configured to receive, via one, some, or all of the plurality of receivers 135A-135N, a plurality of attenuated signals that are ultrasonic signals transmitted by plurality of transmitters 130A-130N after passing through the body 105.

In block 715, the electronic controller 140 determines a phase shift between the ultrasonic signal transmitted by transmitter 130 and the attenuated signal received by the receiver 135, which is the ultrasonic signal after passing through the body 105. For example, the electronic controller 140 may analyze the signals over time to detect the maximum (or peak) of each signal during a period (of the waves), and determine the elapsed time between the peaks of the transmitted ultrasonic signal and the received attenuated ultrasonic signal. In some embodiments, this elapsed time is the determined phase shift. In some embodiments, the phase shift is expressed in terms of an angle, which can be calculated using the equation: phase shift=360*elapsed time between the peaks/period of the transmitted ultrasonic signal. In some embodiments, zero crossings or minimums of each of the two signals may also be used instead of the peaks for these signals. Additionally, in some embodiments, other phase shift analysis techniques are used to determine the phase shift between the transmitted signal and received signal.

For example, with respect to FIGS. 8A and 8B, the electronic controller 140 may be configured to determine a phase shift between the transmitted signal 800A and the received signal 800B. The phase shift is identified in FIG. 8B as “PS NBF,” wherein “PS NBF” refers to the phase shift (PS) between the transmitted signal 800A and the received signal 800B when there is no biofilm (NBF) present on an inner surface 110 of the body 105. As shown, the received signal 800B is delayed, or shifted, by 0.5 microseconds (μs) relative to the transmitted signal 800A. Thus, as the transmitted signal 800A passes through the body 105, the transmitted signal 800A experiences a phase shift of 0.5 μs by the time the transmitted signal 800A is received, as the received signal 800B, by receiver 135. It should be understood that the value, 0.5 μs, of the phase shift between transmitted signal 800A and received signal 800B is provided as an example and that the value of the phase shift between transmitted signal 800A and received signal 800B may change depending on the amplitude of the transmitted signal 800A and/or the type and thickness of material from which body 105 is constructed.

With respect to FIGS. 9A and 9B, the electronic controller 140 may be configured to determine a phase shift between the transmitted signal 900A and the received signal 900B. The phase shift is identified in FIG. 9B as “PS BF,” wherein “PS BF” refers to the phase shift (PS) between the transmitted signal 900A and the received signal 900B when there is biofilm (BF) present on an inner surface 110 of the body 105. As shown, the received signal 900B is delayed, or shifted, by approximately 0.75 μs relative to the transmitted signal 900A. Thus, as the transmitted signal 900A passes through the body 105, the transmitted signal 900A experiences a phase shift of 0.75 μs by the time the transmitted signal 900A is received, as the received signal 900B, by receiver 135.

When compared to the phase shift, PS NBF, that exists between transmitted signal 800A and received signal 800B when no biofilm is present on an inner surface 110 of body 105, the phase shift, PS BF, that exists between transmitted signal 900A and received signal 900B when biofilm is present on an inner surface 110 of body 105 is larger. This can be attributed to additional phase shift induced on transmitted signal 900A by the biofilm as transmitted signal 900A passes through the body 105. For example, with reference to FIG. 4, it will be assumed that the transmitted signal 900A is the ultrasonic signal 165. With reference to FIG. 4, the biofilm 125 present on the inner surface 110 of body 105 may cause an additional shift in the phase of transmitted signal 900A as the transmitted signal 900A passes through the biofilm 125. The additional shift in the phase of transmitted signal 900A caused by biofilm 125 would otherwise not occur if the transmitted signal 900A does not pass through any biofilm (e.g., transmitted signal 800A). In general, the presence of biofilm on an inner surface 110 of body 105 may cause the phase shift between the transmitted signal 900A and the received signal 900B to increase when compared to the phase shift that exists between the transmitted signal 800A and the received signal 800B when there is no biofilm present.

With respect to FIGS. 8B and 9B, the presence of biofilm on an inner surface 110 of the body 105 approximately accounts for an additional 0.25 μs of phase shift, PS BF, between transmitted signal 900A and received signal 900B when compared to the phase shift, PS NBF, between transmitted signal 800A and received signal 800B. However, it should be understood that the value of additional phase shift between transmitted and received signals attributed to the presence of biofilm is provided as an example and that, in other instances, the presence of biofilm may cause phase shifts that are greater than or less than 0.25 μs. In some cases, the amount by which the presence of biofilm contributes to the phase shift between the transmitted signal 900A and received signal 900B may be dependent on the type, thickness, and/or length of the biofilm. For example, biofilm of a first length (e.g., 0.5 mm) may cause a phase shift between the transmitted signal 900A and received signal 900B that is larger than the phase shift between the transmitted signal 900A and received signal 900B that would be caused by biofilm of a second length (e.g. 0.1 mm) that is shorter than the first length. However, regardless of the type, thickness, and/or length of biofilm present on an inner surface 110 of body 105, it can be assumed that the presence of biofilm generally increases the phase shift induced on an ultrasonic signal passing through body 105 when compared to the phase shift induced on an ultrasonic signal passing through body 105 when there is no biofilm present.

Although the above description of block 715 is provided with respect to embodiments in which system 100 includes a transmitter 130 and a receiver 135, it should be understood that the above description is also applicable to embodiments of system 100 in which there are a plurality of transmitter 130A-130N and a plurality of receivers 135A-135N. That is, the electronic controller 140 may be configured to determine a respective phase shift between signals transmitted by each one of a plurality of transmitters 130A-130N and signals received by each one of a plurality of receivers 135A-135N.

In block 720, the electronic controller 140 determines an amplitude difference between the ultrasonic signal transmitted by transmitter 130 and the attenuated ultrasonic signal received by the receiver 135, which is the ultrasonic signal after passing through the body 105. For example, the electronic controller 140 may analyze the transmitted ultrasonic signal and the received attenuated ultrasonic signal over time to detect the maximum (or peak) of each signal during a period (of the waves), determine the amplitude of each peak, and then subtract one amplitude from the other amplitude to determine the amplitude difference.

For example, with respect to FIGS. 8A and 8B, the electronic controller 140 may be configured to determine an amplitude difference between the transmitted signal 800A and the received signal 800B. The amplitude of transmitted signal 800A is identified in FIG. 8A as “Vpp Transmitted NBF,” wherein “Vpp Transmitted NBF” refers to the peak-to-peak amplitude of the transmitted signal 800A when there is no biofilm (NBF) present on an inner surface 110 of the body 105. As shown, transmitted signal 800A has an amplitude of 8 Vₚₚ. Similarly, the amplitude of received signal 800B is identified in FIG. 8B as “Vpp Received NBF,” wherein “Vpp Received NBF” refers to the peak-to-peak amplitude of the received signal 800B when there is no biofilm (NBF) present on an inner surface 110 of the body 105. As shown, received signal 800B has an amplitude of 4 Vₚₚ. Thus, electronic controller 140 would determine that the amplitude difference between the transmitted signal 800A and the received signal 800B is 4 Vₚₚ. As the transmitted signal 800A passes through the body 105, the transmitted signal 800A is attenuated, or reduced in strength, by the time the transmitted signal 800A is received, as the received signal 800B, by receiver 135. That is, when the transmitted signal 800A passes through the body 105, the amplitude of the transmitted signal 800A is decreased. It should be understood that the value, 4 Vₚₚ, of the amplitude difference between transmitted signal 800A and received signal 800B is provided as an example and that the value of the amplitude difference between transmitted signal 800A and received signal 800B may change depending on the amplitude of the transmitted signal 800A, the frequency of the transmitted signal 800A, and/or the type and thickness of material from which body 105 is constructed.

With respect to FIGS. 9A and 9B, the electronic controller 140 may be configured to determine an amplitude difference between the transmitted signal 900A and the received signal 900B. The amplitude of transmitted signal 900A is identified in FIG. 9A as “Vpp Transmitted BF,” wherein “Vpp Transmitted BF” refers to the peak-to-peak amplitude of the transmitted signal 900A when there is biofilm (BF) present on an inner surface 110 of the body 105. As shown, transmitted signal 900A has an amplitude of 8 Vₚₚ. Similarly, the amplitude of received signal 900B is identified in FIG. 9B as “Vpp Received BF,” wherein “Vpp Received BF” refers to the peak-to-peak amplitude of the received signal 900B when there is biofilm (BF) present on an inner surface 110 of the body 105. As shown, received signal 900B has an amplitude of 3.6 Vₚₚ. Thus, electronic controller 140 would determine that the amplitude difference between the transmitted signal 900A and the received signal 900B is 4.4 Vₚₚ. As the transmitted signal 900A passes through the body 105, including the biofilm (e.g., biofilm 125) present on the inner surface 110 of body 105, the transmitted signal 900A is reduced in strength by the time the transmitted signal 900A is received, as received signal 800B, by receiver 135. That is, when the transmitted signal 900A passes through the body 105, the amplitude of the transmitted signal 900A is decreased. It should be understood that the value, 4.4 Vₚₚ, of the amplitude difference between transmitted signal 900A and received signal 900B is provided as an example and that the value of the amplitude difference between transmitted signal 900A and received signal 900B may change depending on the amplitude of the transmitted signal 900A, the frequency of the transmitted signal 900A, an amount of biofilm present on an inner surface 110 of body 105, and/or the type and thickness of material from which body 105 is constructed.

When compared to the amplitude difference that exists between transmitted signal 800A and received signal 800B when no biofilm is present on an inner surface 110 of body 105, the amplitude difference that exists between transmitted signal 900A and received signal 900B when biofilm is present on an inner surface 110 of body 105 is larger. This can be attributed to additional attenuation of transmitted signal 900A caused by the biofilm as the transmitted signal 900A passes through the body 105. For example, with reference to FIG. 4, it will be assumed that the transmitted signal 900A is the ultrasonic signal 165. With reference to FIG. 4, the biofilm 125 present on the inner surface 110 of body 105 may cause additional attenuation, or reduction in amplitude, of transmitted signal 900A as the transmitted signal 900A passes through the biofilm 125. In general, the presence of biofilm on an inner surface 110 of body 105 may cause the amplitude difference between the transmitted signal 900A and the received signal 900B to increase when compared to the amplitude difference between the transmitted signal 800A and the received signal 800B when there is no biofilm present.

With respect to FIGS. 8-9, the presence of biofilm on an inner surface 110 of the body 105 accounts for an additional 0.4 Vₚₚ of amplitude difference between transmitted signal 900A and received signal 900B when compared to the amplitude difference between transmitted signal 800A and received signal 800B. However, it should be understood that the value of additional amplitude difference between transmitted and received signals attributed to the presence of biofilm is provided as an example and that, in other instances, the presence of biofilm may cause additional amplitude differences that are greater than or less than 0.4 Vₚₚ. In some cases, the amount by which the presence of biofilm contributes to the amplitude difference between the transmitted signal 900A and received signal 900B may be dependent on the type, thickness, and/or length of the biofilm. For example, biofilm of a first length (e.g., 0.5 mm) may cause an additional amplitude difference between the transmitted signal 900A and received signal 900B that is larger than the additional amplitude difference between the transmitted signal 900A and received signal 900B that would be caused by biofilm of a second length (e.g. 0.1 mm), the second length being shorter than the first length. However, regardless of the type, thickness, and/or length of biofilm present on an inner surface 110 of body 105, it can be assumed that the presence of biofilm generally increases the amount of attenuation, or amount by which an amplitude is reduced, of on an ultrasonic signal passing through body 105 when compared to the attenuation of an ultrasonic signal passing through body 105 when there is no biofilm present.

Although the above description of block 725 is provided with respect to embodiments in which system 100 includes a transmitter 130 and a receiver 135, it should be understood that the above description is also applicable for embodiments of system 100 in which there are a plurality of transmitter 130A-130N and a plurality of receivers 135A-135N. That is, the electronic controller 140 may be configured to determine a respective amplitude difference between signals transmitted by each one of a plurality of transmitters 130A-130N and signals received by each one of a plurality of receivers 135A-135N.

In block 725, the electronic controller 140 generates an indication of an amount of biofilm present on an inner surface 110 of the body 105 based on the phase shift and the amplitude difference between the transmitted ultrasonic signal and the received ultrasonic signal.

In some embodiments, the electronic controller 140 determines the amount of biofilm present by calculating the amount of biofilm using a function. In such embodiments, the function may be a function in which the phase shift and amplitude difference between the transmitted ultrasonic signal and the received ultrasonic signal are inputs and the amount of biofilm is an output. The function may, for example, define a relationship in which the amount of biofilm present increase and the phase shift and amplitude difference between the transmitted ultrasonic signal and the received ultrasonic signal increases. For example, when the electronic controller 140 uses the function to calculate an amount biofilm present on an inner surface 110 of body 105 when the phase shift and amplitude difference between transmitted signal 800A and received signal 800B are provided as inputs, the output of the function may indicate that there is no biofilm present on the inner surface 110 of body 105. As another example, when the electronic controller 140 uses the function to calculate an amount biofilm present on an inner surface 110 of body 105 when the phase shift and amplitude difference between transmitted signal 900A and received signal 900B are provided as inputs, the output of the function may indicate the amount of biofilm present by indicating one or both of (i) whether biofilm is present on the inner surface 110 of the body 105 (e.g., a binary indication that there is biofilm of a non-zero thickness, length, and/or concentration is present on the inner surface 110) and (ii) the particular thickness, length, and/or concentration of biofilm that is present on the inner surface 110.

In some embodiments, the material type and/or thickness from which body 105 is formed may additionally be provided as inputs to the function. Accordingly, in such embodiments, the electronic controller 140 is operable to further determine an amount of biofilm present based on the material type and/or thickness of body 105 in addition to the phase shift and amplitude difference between the transmitted ultrasonic signal and the received ultrasonic signal. As described above, the type and/or thickness of material from which body 105 is formed affects the amount of amplitude difference and/or phase shift that occurs between the transmitted ultrasonic signal and the received ultrasonic signal. Thus, the electronic controller 140 may be configured to accurately determine the amount of biofilm present on an inner surface 110 of the body 105, regardless of the material from which body 105 is formed.

In some embodiments, one or more of the functions noted herein are generated from analysis of test data generated by testing bodies having different shapes, thicknesses, and materials with known biofilm thicknesses (e.g., test inputs and output are plotted as data points and the function represents a best fit line or curve for the plotted data points).

In some embodiments, the electronic controller 140 determines the amount of biofilm present on an inner surface 110 of body 105 by accessing a lookup table that defines a relationship between the amount of biofilm present and at least one of the phase shift and amplitude difference between the transmitted ultrasonic signal and the received ultrasonic signal. In some embodiments, the electronic controller 140 determines the amount of biofilm present on an inner surface 110 of body 105 by accessing a lookup table that defines a relationship between the amount of biofilm present, the type and/or thickness of material from which the body 105 is formed, and at least one of the phase shift and amplitude difference between the transmitted ultrasonic signal and the received ultrasonic signal. For example, when the electronic controller 140 accesses a look-up table to determine an amount of biofilm present on an inner surface 110 of body 105, the look-up table may indicate that there is no biofilm present when the phase shift and/or amplitude difference between the transmitted ultrasonic signal are respectively less than or equal to the phase shift and/or amplitude difference between transmitted signal 800A and received signal 800B. As another example, when the electronic controller 140 accesses a look-up table to determine an amount biofilm present on an inner surface 110 of body 105, the look-up table may indicate that a non-zero amount (e.g., length, thickness, and/or concentration) of biofilm is present when the phase shift and/or amplitude difference between the transmitted ultrasonic signal are approximately equal to the phase shift and/or amplitude difference between transmitted signal 900A and received signal 900B. In some embodiments, the lookup table output provides a particular thickness, length, and/or concentration value indicative of the amount of biofilm present on the inner surface 110.

As noted, the functions and lookup tables may provide the indication of the amount of biofilm present on the inner surface 110 by providing a binary indication of whether biofilm is present, by providing a particular thickness, length, and/or concentration value for the biofilm present, or by providing both the binary indication and the particular value(s). In some embodiments, the indication of the amount of biofilm present on the inner surface 110 of body 105 further includes a classification of the type of biofilm present on the inner surface 110. In such embodiments, the electronic controller 140 determines the classification of biofilm based on at least one of the phase shift and amplitude difference between the transmitted ultrasonic signal and the received ultrasonic signal. For example, the electronic controller 140 may use a function or lookup table, generated based on test data in a similar manner as described above, that maps one or more of the phase shift, amplitude difference, and the type and thickness of the body 105 to the classification of the biofilm.

In some embodiments, the indication of the amount of biofilm present on the inner surface 110 of the body 105 may include a message to an operator of the system 100. In such embodiments, the message may include an amount of biofilm present, a suggested action based on the amount of biofilm present, and/or an approximate location of the biofilm that is present. For example, the indication of the amount of biofilm present may include a maintenance warning that indicates the body 105 should be cleaned, replaced, or otherwise serviced. As another example, the indication of the amount of biofilm present may include an estimated time and/or date by which the body 105 should be cleaned, replaced, or otherwise serviced. As another example, the indication of the amount of biofilm present may include an indication that the body 105 is safe to operate (e.g., that there is not a harmful amount of biofilm present).

In some embodiments, the electronic controller 140 may be configured to generate the indication of the amount of biofilm present on the inner surface 110 of the body 105 based on whether the phase shift between the transmitted ultrasonic signal and the received ultrasonic signal exceeds a first threshold and/or based on whether the amplitude difference between the transmitted ultrasonic signal and the received ultrasonic signal exceeds a second threshold. For example, the first threshold and/or second thresholds may be associated with an amount of biofilm present on the inner surface 110 of body 105 that is considered to be a dangerous, harmful, or otherwise undesirable amount of biofilm. Thus, the electronic controller 140 may be configured to generate the indication in response to determining that at least one the phase shift between the transmitted ultrasonic signal and the received ultrasonic signal exceeds a first threshold and/or the amplitude difference between the transmitted ultrasonic signal and the received ultrasonic signal exceeds a second threshold.

In some embodiments, a value for the first threshold is determined during a calibration process. For example, the calibration process may include determining a baseline phase shift, which is the phase shift between the transmitted ultrasonic signal and the received ultrasonic signal at an initial time, such as a time at which it is known that no or minimal biofilm is present on the inner surface 110 of body 105 (e.g., after a cleaning of the body 105 or at initial installation of the body 105). Accordingly, the value of the first threshold may be chosen to be a phase shift value that is greater (e.g., by a predetermined or user-specified amount) than the phase shift that exists between the transmitted ultrasonic signal and the received ultrasonic signal when there is no biofilm present. In some embodiments, a value for the first threshold is provided as a user input, via the user-interface 615, to the electronic controller 140. In some embodiments, a value for the first threshold is provided by an external device to the electronic controller 140 via the communication circuit 620.

Similarly, in some embodiments, a value for the second threshold is determined during a calibration process. For example, the calibration process may include determining a baseline amplitude difference, which is the amplitude difference between the transmitted ultrasonic signal and the received ultrasonic signal at the initial time, such as a time at which it is known that no or minimal biofilm is present on the inner surface 110 of body 105. Accordingly, the value of the second threshold may be chosen to be an amplitude difference value that is greater (e.g., by a predetermined or user-specified amount) than the amplitude difference that exists between the transmitted ultrasonic signal and the received ultrasonic signal when there is no biofilm present. In some embodiments, a value for the second threshold is provided as a user input, via the user-interface 615, to the electronic controller 140. In some embodiments, a value for the second threshold is provided by an external device to the electronic controller 140 via the communication circuit 620.

In some embodiments, the electronic controller 140 receives user input (e.g., via the user-interface 615 or an external device) that specifies characteristics of the application in which the system 100 is to be used (application characteristics). The application characteristics may indicate one or more of the material, thickness, or shape of the body 105, the type of biofilm to be detected, and a sensitivity level (e.g., low, medium, or high) desired for the biofilm detection. The electronic controller 140 may then access a lookup table (e.g., in the memory 150) with the application characteristics as input, and receive the first threshold, the second threshold, or both the first and second thresholds as outputs.

In some embodiments, the electronic controller 140 provides the indication of an amount of biofilm present on the inner surface 110 of the body 105 to an operator of system 100 via the user-interface 615. For example, the indication may be a visual indication displayed, by the electronic controller 140, on a display of the user-interface 615. As another example, the indication may be an audible indication transmitted by a speaker of the user-interface 615. In some embodiments, the electronic controller 140 is configured to provide the indication, via the communication circuit 620, to an external device. In such embodiments, the indication may be a visual indication displayed on a display of the external device and/or an audible indication transmitted by a speaker of the external device.

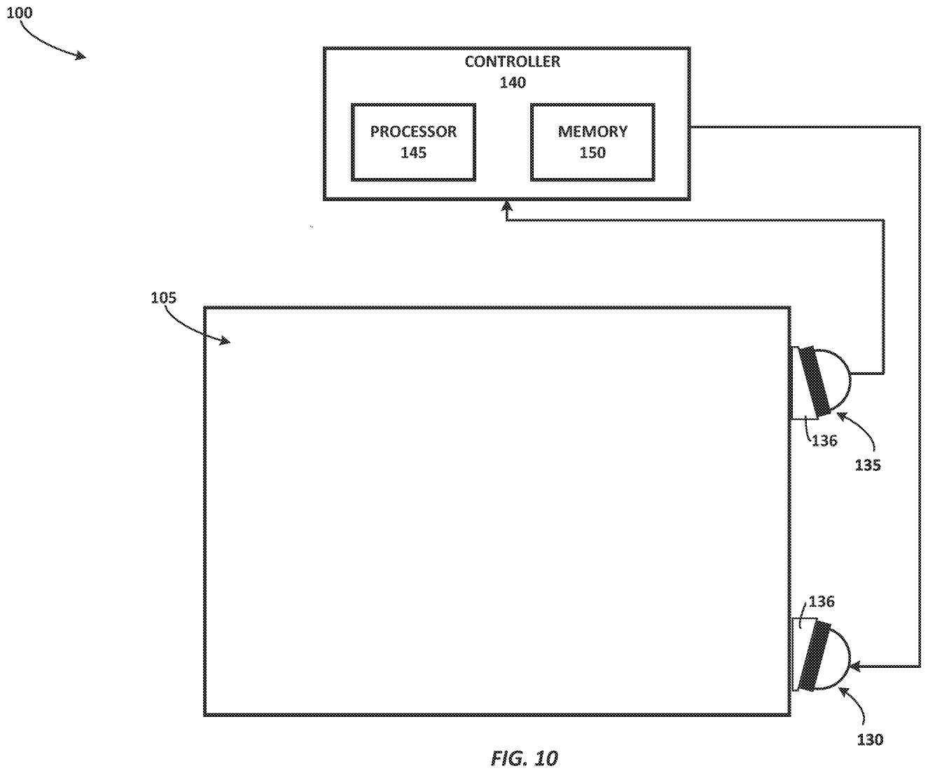

FIG. 10 illustrates a schematic view of an embodiment of the system 100 in which the transmitter 130 and the receiver 135 are located in positions external to, and on the same side of, the body 105. For example, with respect to FIG. 10, the transmitter 130 and the receiver 135 are each located in a respective position external to the right side of the body 105. When the transmitter 130 and the receiver 135 are located in positions external to the same side of body 105, ultrasonic signals transmitted by the transmitter 130 are reflected off an inner surface of the body 105 towards the receiver 135. In some embodiments of the system 100, an impedance matching material 136 fills the gap between the transducer elements 130, 135 and the body 105. The impedance matching material 136 may be in the form of an impedance matching wedge, and is herein referred to as the wedge 136. The wedge may be made of any material with impedance matching characteristics, such as a polymer matching material. During transmission, the transmitted signal enters the wedge 136 at a right angle while the adjusted penetration angle to the body does not change. Upon reception, the received signal will again enter the receiver 135 at a right angle from the wedge 136 while the adjusted reception angle from the body 105 remains unchanged.

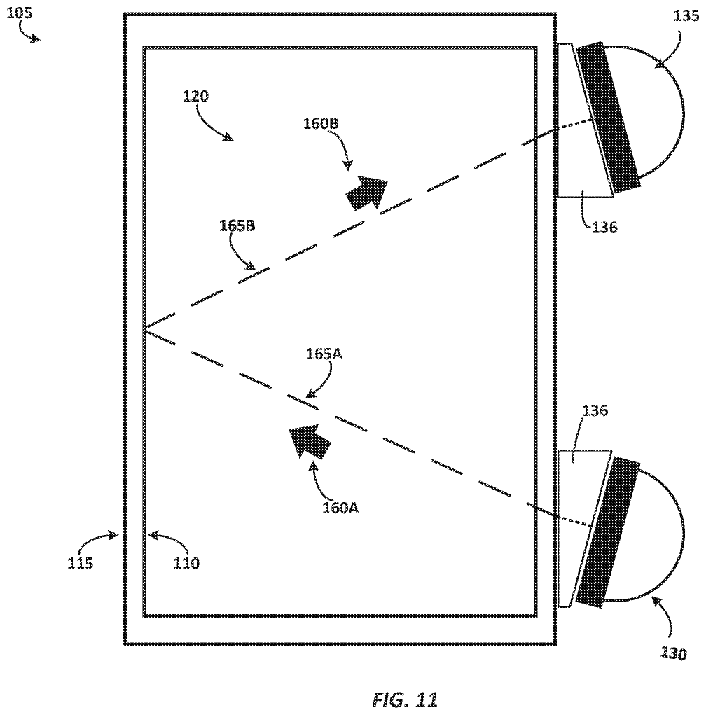

FIG. 11 illustrates a cutaway view of the system 100 illustrated in FIG. 10. As shown, the transmitter 130 transmits an ultrasonic signal 165A in a direction (indicated by arrow 160A in FIG. 11) towards the body 105, such that the ultrasonic signal 165A enters the body 105 through the outer surface 115 of the right side of body 105. The ultrasonic signal 165 is then reflected off of an inner surface 110 of the left side of the body 105, such that a reflected version of the ultrasonic signal 165A, reflected signal 165B, travels in a direction (indicated by arrow 160B in FIG. 11) towards the right side of the body 105. The reflected signal 165B exits the body 105 through the outer surface 115 of the right side of the body 105 and is received by receiver 135. Using any of the methods and processes described herein (e.g., the method 700 of FIG. 7), the electronic controller 140 is then configured to detect the presence of and/or determine an amount of biofilm present on an inner surface of the body 105 based on a phase shift between the transmitted ultrasonic signal 165A and the reflected ultrasonic signal 165B, an amplitude difference between the transmitted ultrasonic signal 165A and the reflected ultrasonic signal 165B, and/or the type of material from which body 105 is constructed. Accordingly, FIGS. 10 and 11 illustrate an embodiment of system 100 in which transmitter(s) 130 and receiver(s) 135 located at positions external to the same side of body 105 can be used to detect the presence of biofilm on an inner surface of the body 105.

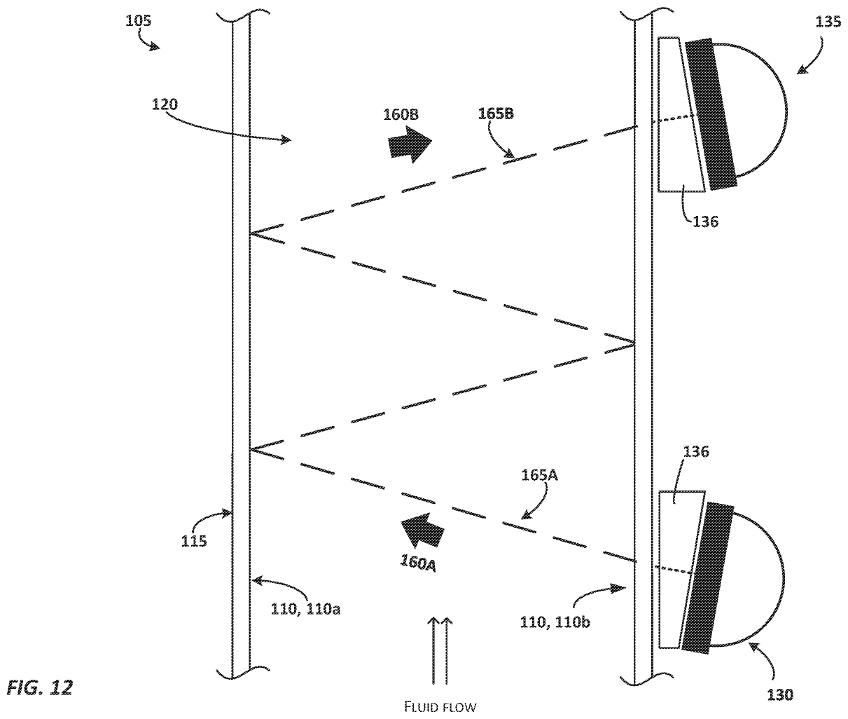

In some embodiments, such as illustrated in FIG. 12, the transmitter 130 and receiver 135 are spaced apart along the exterior of body 105 such that the ultrasonic signal 165A is reflected multiple times off of the inner surfaces of body 105 before a reflected version of the ultrasonic signal 165A is received. For example, although FIG. 11 illustrates the ultrasonic signal 165A as being reflected a single time before the reflected signal 165B is received by the receiver 135, in some embodiments, the ultrasonic signal 165A is reflected off of the inner surfaces of body 105 multiple times (e.g., five times, ten times, etc.) before a reflected version of the signal 165A is received by the receiver 135. In FIG. 12, the body 105 is illustrated as a cross section of a pipe through which a fluid flows. However, the sensor arrangement described with respect to FIG. 12 is also applicable to the other types of bodies that the body 105 may take. Additionally, although the ultrasonic signal 165A is illustrated as being reflected three times within the body 105 by the inner surface 110, in other embodiments, more reflections occur, such as by adjusting the angle of the wedge 136, the angle of the transmitter 130, the width of the body 105, and the spacing between the transmitter 130 and the receiver 135.

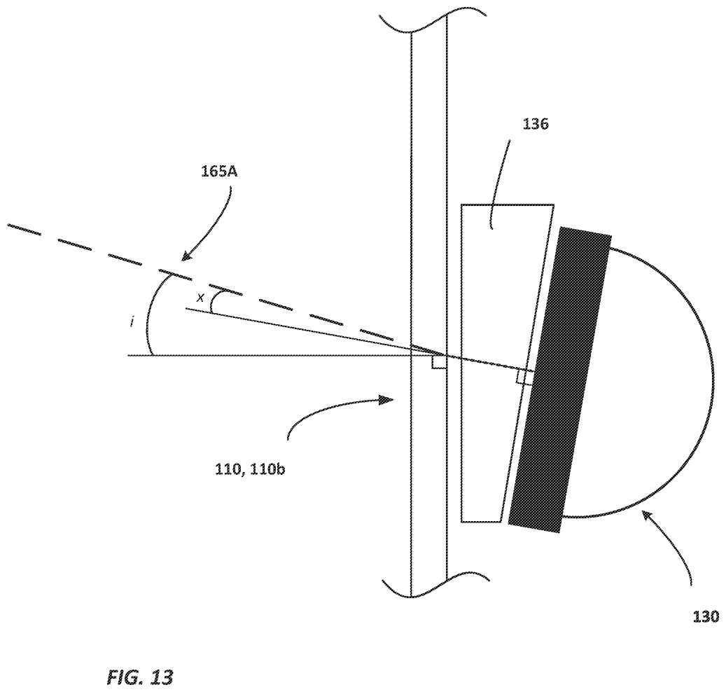

In some embodiments, the transmitter 130 and wedge 136 are arranged such that the angle of the signal 165 relative to the normal of the surface 110 (the incident angle i, FIG. 13) exceeds the critical angle. The critical angle is the angle of incidence at which the signal is completely reflected from the boundary, and where total internal reflection takes place. Accordingly, in such embodiments where the incident angle exceeds the critical angle, the total reflection of the signal 165 will occur at the inner surface 110 of the body 105, which may include a first reflection surface 110a and a second reflection surface 110b. When the first and second reflection surfaces 110a-b are arranged parallel to each other, they form an acoustic waveguide.

When the ultrasonic signal 165 enters the waveguide (i.e., the body 105) at an angle greater than the critical angle, the signal will be reflected repeatedly back and forth between the first and second reflection surfaces 110a-b, and eventually will exit the waveguide (the body 105) and be received by the receiver 135. In such embodiments, attenuation of the ultrasonic signal 165A accrues each time the ultrasonic signal 165A is reflected. Thus, the ultrasonic signal 165A may need to be transmitted by the transmitter 130 at higher power level or amplitude to account for the attenuation, so that the reflected signal is not too far diminished to be analyzed at the point it is received by the receiver 135. Additionally or alternatively, to account for the accrued attenuation from multiple reflections, additional amplification of the reflected version of the ultrasonic signal 165A may be required before the reflected version of the ultrasonic signal 165A is used by the electronic controller 134 to determine an amount of biofilm present on an inner surface of the body 105. However, because of the multiple reflections, the accrued attenuation and phase shift provided by each pass through the biofilm may enable the electronic controller 134 to more easily detect the impact of the biofilm on the ultrasonic signal 165a and, thus, characterize the biofilm.

FIG. 13 illustrates a close-up view of the ultrasonic signal 165 of FIG. 12. As shown, the ultrasonic signal 165 is transmitted at an incident angle 165 through the body 105. As also shown, the ultrasonic signal 165 enters the wedge 136 at a right angle, and this right angle varies from the incident angle by an angle x.

Accordingly, embodiments described herein provide methods and systems for detecting and generating an indication of a biofilm on a body, such as a pipe, tank, or other container. Various features and advantages of some embodiments are set forth in the following claims.

What is claimed is:

1. A system for detecting the presence of biofilm on an inner surface of a body used for containing a fluid medium comprising:
a transmitter disposed at a first location external to the body;
a receiver disposed at a second location external to the body; and
an electronic controller configured to:
control the transmitter to transmit an ultrasonic signal in a direction towards the body;
receive, via the receiver, an attenuated ultrasonic signal that is the ultrasonic signal after passing through the body;
determine a phase shift between the ultrasonic signal and the attenuated ultrasonic signal;
determine an amplitude difference between the ultrasonic signal and the attenuated ultrasonic signal; and
generate an indication of an amount of biofilm present on the inner surface of the body based on the phase shift and the amplitude difference.

2. The system of claim 1, wherein the body is one selected from the group consisting of a pipe, a tank, a flexible container, and a flexible tube.

3. The system of claim 1, wherein the first location is disposed at a location opposite the second location relative to the body.

4. The system of claim 1, wherein the first location and the second location are positioned on a same side of the body, and the ultrasonic signal is reflected by the inner surface of the body at least once before reaching the receiver as the attenuated ultrasonic signal.

5. The system of claim 1, wherein, to calibrate the system, the electronic controller is further configured to:
determine a baseline phase shift between the ultrasonic signal and the attenuated ultrasonic signal at an initial time; and
determine a baseline amplitude difference between the ultrasonic signal and the attenuated ultrasonic signal at the initial time.

6. The method of claim 1, wherein the amplitude of the ultrasonic signal transmitter is less than 15 V and the amplitude of the attenuated signal at the receiver is greater than 200 mV.

7. The system of claim 1, wherein the electronic controller is further configured to generate the indication of the amount of biofilm present on the inner surface of the body based on the phase shift and the amplitude difference in response to at least one selected from the group of:
determining whether the phase shift between the ultrasonic signal and the attenuated signal exceeds a first threshold; and
determining whether the amplitude difference between the ultrasonic signal and the attenuated signal exceeds a second threshold.

8. The system of claim 7, wherein the electronic controller is further configured to:
determine the first threshold by at least one selected from the group of a calibration process and a communication from a user device; and
determine the second threshold by at least one selected from the group of a calibration process and a communication from a user device.

9. The system of claim 1, wherein the electronic controller is further configured to determine the amount of biofilm present on the inner surface of the body by at least one selected from the group of:
calculating the amount of biofilm present using a function in which the phase shift and amplitude difference are inputs and the amount of biofilm is an output, where the function defines a relationship in which the amount of biofilm present increases as the phase shift and the amplitude difference increase; and
accessing a lookup table that defines a relationship between the amount of biofilm present and at least one selected from the group of the phase difference and the amplitude.

10. The system of claim 9, wherein the electronic controller is further configured to:
determine whether the amount exceeds a first threshold, wherein the electronic controller generates the indication of the amount of biofilm present on the inner surface of the body in response to determining that the amount exceeds the first threshold.

11. A method of detecting the presence of biofilm on an inner surface of a body used for containing a fluid medium, the method comprising:
controlling, by an electronic controller, a transmitter disposed at a first location external to the body to transmit an ultrasonic signal in a direction towards the body;
receiving, by the electronic controller, via a receiver disposed at a second location external to the body, an attenuated ultrasonic signal that is the ultrasonic signal after passing through the body;
determining, by the electronic controller, a phase shift between the ultrasonic signal and the attenuated ultrasonic signal;
determining, by the electronic controller, an amplitude difference between the ultrasonic signal and the attenuated ultrasonic signal; and
generating, by the electronic controller, an indication of an amount of biofilm present on the inner surface of the body based on the phase shift and the amplitude difference.

12. The method of claim 11, wherein the body is one selected from the group consisting of a pipe, a tank, a flexible container, and a flexible tube.

13. The method of claim 11, further comprising:
performing, by the electronic controller, calibration including:
determining a baseline phase shift between the ultrasonic signal and the attenuated ultrasonic signal at an initial time; and
determining a baseline amplitude difference between the ultrasonic signal and the attenuated ultrasonic signal at the initial time.

14. The method of claim 11, wherein the ultrasonic signal has a frequency that is less than 5 Mhz

15. The method of claim 11, wherein the amplitude of the ultrasonic signal transmitter is less than 15 V and the amplitude of the attenuated signal at the receiver is greater than 200 mV.

16. The method of claim 11, wherein generating, by the electronic controller, the indication of the amount of biofilm present on the inner surface of the body based on the phase shift and the amplitude difference includes at least one selected from the group of:
determining, by the electronic controller, whether the phase shift between the ultrasonic signal and the attenuated signal exceeds a first threshold and, in response, generating the indication; and
determining, by the electronic controller, whether the amplitude difference between the ultrasonic signal and the attenuated signal exceeds a second threshold and, in response, generating the indication.

17. The method of claim 16, further comprising:
determining, by the electronic controller, the first threshold by at least one selected from the group of a calibration process and a communication from a user device; and
determining, by the electronic controller, the second threshold by at least one selected from the group of a calibration process and a communication from a user device

18. The method of claim 11, wherein generating, by the electronic controller, the indication of the amount of biofilm present on the inner surface of the body based on the phase shift and the amplitude difference includes determining the amount of biofilm present on the inner surface of the body by at least one selected from the group of:
calculating, by the electronic controller, the amount of biofilm present using a function in which the phase shift and amplitude difference are inputs and the amount of biofilm is an output, where the function defines a relationship in which the amount of biofilm present increases as the phase shift and the amplitude difference increase; and
accessing, by the electronic processor, a lookup table that defines a relationship between the amount of biofilm present and at least one selected from the group of the phase difference and the amplitude.

19. The method of claim 18, further comprising:
determining, by the electronic controller, whether the amount exceeds a first threshold, wherein the electronic controller generates the indication of the amount of biofilm present on the inner surface of the body in response to determining that the amount exceeds the first threshold.

20. The method of claim 11, wherein the indication of the amount of biofilm present includes at least one selected from the group of:
a thickness value;
a classification of the amount of the biofilm present; and
a maintenance warning to a user.
